SEQUENCE LISTING (13 sequences)

SEQ ID NO: 1 - RNA, 13 nt, Artificial Sequence
  KOZAK SEQUENCE
gccgccrccaugg

SEQ ID NO: 2 - RNA, 64 nt, ARTIFICIAL SEQUENCE
  KOZAK SEQUENCE
gaaagaaagaaagaaagaaagaaagaaagaaagaaagaaagaaagaaagaaagaaagaaagaaa

SEQ ID NO: 3 - RNA, 75 nt, ARTIFICIAL SEQUENCE
  crTMV IRES
uucguuugcuuuuuguaguauaauuaaauauuugucauauaagagauugguuagagauuuguucuuuguuugauc

SEQ ID NO: 4 - RNA, 54 nt, ARTIFICIAL SEQUENCE
  LINE1 IRES
cgcauuaucucuccacgaauccagcccuucaaaggauaauaacagaaaaaaacg

SEQ ID NO: 5 - RNA, 24 nt, ARTIFICIAL SEQUENCE
  RBM3 IRES
uuuauaauuucuucuuccagaauc

SEQ ID NO: 6 - DNA, 56 bp, ARTIFICIAL SEQUENCE
  Apaf-1 IRES
taggcgcaaaggcttggctcatggttgacagctcagagagagaaagatctgaggga

SEQ ID NO: 7 - DNA, 48 bp, ARTIFICIAL SEQUENCE
  c-MYC IRES
ggggactttgcactggaacttacaacacccgagcaaggacgcgactct

SEQ ID NO: 8 - protein, 8 aa, ARTIFICIAL SEQUENCE
  SIINFEKL ANTIGEN
SIINFEKL

SEQ ID NO: 9 - RNA, 27 nt, ARTIFICIAL SEQUENCE
  SIINFEKL CODING REGION
gagaguauaaucaacuuugaaaaacug

SEQ ID NO: 10 - RNA, 10 nt, ARTIFICIAL SEQUENCE
  KOZAK SEQUENCE
gccaccaugg

SEQ ID NO: 11 - protein, 8 aa, ARTIFICIAL SEQUENCE
  FLAG tag
DYKDDDDK

SEQ ID NO: 12 - RNA, 56 nt, ARTIFICIAL SEQUENCE
  Apaf-1 IRES
uaggcgcaaaggcuuggcucaugguugacagcucagagagagaaagaucugaggga

SEQ ID NO: 13 - RNA, 48 nt, ARTIFICIAL SEQUENCE
  c-MYC
ggggacuuugcacuggaacuuacaacacccgagcaaggacgcgacucu

(19) United States
(12) Patent Application Publication
(10) Pub. No.: US 2024/0299520 A1
(43) Pub. Date: Sep. 12, 2024

(54) MINI CIRCULAR RNA THERAPEUTICS AND VACCINES AND METHODS OF USE THEREOF

(71) Applicant: VIRGINIA COMMONWEALTH UNIVERSITY, Richmond, VA (US)

(72) Inventors: Guizhi ZHU, Richmond, VA (US); Yu ZHANG, Richmond, VA (US)

(21) Appl. No.: 18/276,140

(22) PCT Filed: Feb. 8, 2022
(86) PCT No.: PCT/US2022/015604

§ 371 Date: Aug. 7, 2023

Related U.S. Application Data
(60) Provisional application No. 63/147,371, filed on Feb. 9, 2021.
(60) Provisional application No. 63/186,899, filed on May 11, 2021.

(51) Int. Cl. A61K 39/00; A61K 45/06; A61P 35/00; C12N 15/66

(52) U.S. Cl. CPC A61K 39/00119; A61K 39/001164; A61K 45/06; A61P 35/00; C12N 15/66; C12Y 101/01042; A61K 2039/53; A61K 2039/55555; A61K 2039/572

(57) ABSTRACT

Synthetic mini circular RNA vaccine constructs are provided. The synthetic mini circular RNA constructs encode one or more antigens and are used, for example, as vaccines against cancer or infectious agents. In some aspects, the one or more antigens are translated as concatemer peptides by rolling cycle translation (RCT) of the mini circular RNA.

### CROSS-REFERENCE TO RELATED APPLICATIONS

This application claims benefit of U.S. Provisional Patent Applications No. 63/147,371 filed Feb. 9, 2021, and 63/186,899 filed May 11, 2021.

### SEQUENCE LISTING

This application includes as the Sequence Listing the complete contents of the accompanying text file “Sequence.txt”, created Feb. 3, 2022, containing 5 kilobytes, hereby incorporated by reference.

### BACKGROUND OF THE INVENTION

### Field of the Invention

The invention generally relates to synthetic mini circular RNA constructs. In particular, the invention provides synthetic mini circular RNA constructs that are used, for example, as vaccines.

### Description of Related Art

Vaccination has had a tremendous impact on global health and the quality of human life by preventing more than 20 life-threatening diseases. Immunization currently prevents 2-3 million deaths every year from diseases like diphtheria, tetanus, pertussis, influenza and measles. Up to now, the vaccines can be prophylactic or therapeutic and can broadly be classified as live attenuated vaccines (weakened microorganisms), inactivated vaccines (killed microorganisms), subunit vaccines (purified antigens), or toxoid vaccines (inactivated bacterial toxins). As opposed to the conventional concept of injecting live-attenuated or inactivated pathogens, modern vaccine approaches, i.e., subunit vaccines that cover antigen epitopes are attractive due to the ease of large-scale manufacture, storage and transportation without cold chains, long shelf-lives, and good safety. However, subunit antigens often display lower immunogenicity, which can be rectified by employing delivery systems and/or immunopotentiating compounds as adjuvants to boost immunogenicity.

Nucleic acid-based vaccines, i.e., DNA (as plasmids) and RNA (as messenger RNA (mRNA)) vaccines, pave the way for safe and efficacious biologics to mimic inoculation with live organism-based vaccines, particularly for stimulation of cell-mediated immunity. Within nucleic acid-based vaccines, the emerging mRNA vaccines have several notable features. 1) mRNA avoids the risk of genomic integration such as that which is a safety concern for the counterpart DNA vaccines, and a low risk of iatrogenic viral infection compared to inactivated viruses or viral vectors. 2) Vector introduction is not limited by potential for pre-existing immunity such as that with a vector-based vaccine approach. 3) mRNA is metabolically degradable, which avoids concerns over the long-term safety. 4) mRNA vaccines have the potential for broad application by encoding any antigen of interest. 5) In contrast to physiochemically heterogeneous peptide vaccines which demand customized formulation, mRNA formulation is generally consistent. Despite these potential advantages, there are key challenges that have impeded the successful translation of mRNA for all applications. For example, (i) mRNA is a very large molecule, (ii) it is intrinsically unstable and prone to degradation by nucleases, and (iii) intracellular mRNA levels, and consequently, antigen translation, has been limited by the short half-life and biostability of mRNA and cellular division.

Recent advances of nucleic acid-based vaccines into human clinical trials has demonstrated proof of principle for the potential of vaccines with mRNA transfected dendritic cells (DCs) targeting tumor antigens as an effective strategy for the treatment of cancer. Today, clinical trials with direct administration of synthetic mRNA encoding tumor antigens have already demonstrated safety, induction of tumor-specific immune responses and the potential for clinical benefit for patients. Furthermore, the importance of the intrinsic self-adjuvanting effects of mRNA is now clearly recognized as a key to the successful implementation of this approach for vaccination. RNA inherently induces immune stimulation by activating pattern recognition receptors, the natural role of which is to identify and respond to viral RNAs with downstream activation of an innate immune response. In immune cells, the Toll-like receptors TLR3, TLR7 and TLR8, which reside in the endosomal compartment, are activated by endocytosed RNA and induce secretion of interferons. By contrast, most of the interferon production in non-immune cells is induced by the activation of two cytosolic receptors: cytoplasmic retinoic acid-inducible gene I protein (RIG-I; also known as DDX58) and melanoma differentiation-associated protein 5 (MDA5; also known as IFIH1).

Beyond cancer, vaccines have quite obviously been a monumental advance for the public health management of infectious diseases, and there is no better example than the recent development of highly effective vaccines to prevent COVID-19, caused by the pandemic virus severe acute respiratory syndrome coronavirus 2 (SARS-CoV-2). The pandemic variants of SARS-CoV-2 mainly attack the lower respiratory system, and infect the gastrointestinal system, heart, kidney, liver, and central nervous system, leading to multiple organ failure. Vaccines have made a huge impact in reducing symptomatic infection and severe disease, which has lessened the burden on hospitals during deadly pandemic surges. Two mRNA vaccine candidates have been amongst the earliest and most effective vaccines implemented during the pandemic, taking advantage of the versality of mRNA as a vaccine strategy which allows for rapid development. The mRNA vaccines against COVID-19 developed by Moderna/NIAID (mRNA-1273) and BioNTech/Pfizer (BNT162b2) demonstrated >90% efficacy in prevention of symptomatic COVID-19 with a good safety profile, and, thus, billions of vaccine doses have been administered worldwide. The same mRNA vaccine technology from the pandemic is also being employed in peptide cancer vaccines targeting some of the most deadly and hard-to-treat cancers, such as melanoma, lung cancer, and colorectal cancer, just to name a few. The early signs from clinical trials suggest that peptide cancer vaccines are well tolerated and could be an important tool for the future of cancer treatment, both in early and late-stage cancers.

Despite the early success of RNA vaccine technology with the pandemic and early-stage cancer vaccines, the initial approach with mRNA vaccines has not been optimized and there will likely be generational improvements as scientists continue to make new discoveries in this young area of scientific interest. Traditional mRNA vaccines must be transcribed using a cloned DNA template which can introduce challenges with rapid customization and large-scale production. Even with synthetic modifications, mRNA vaccines have strict requirements for storage at low temperatures to avoid degradation and, even more importantly, have a short half-life within antigen presenting cells in the body. Limited biostability means that production of the target antigen and antigen exposure will end quickly and limit the potential for immune activation. The inherent instability of mRNA also requires that mRNA vectors be designed with large non-coding sequences that simply allow the structure to be more stable, and also help position the mRNA for recruitment of ribosomes for protein synthesis. The drawback of mRNA requiring such a large sequence means that less of the vector encodes the target antigen sequence, and therefore fewer copies of antigen encoding RNA can fit into each lipid nano-carrier for delivery to antigen presenting cells to be translated into the antigen of interest. Additionally, RNA and its lipid nanocarriers can both inherently activate innate immunity; overstimulation of innate immunity can reduce antigen expression and immunogenicity, and also result in poor tolerability. Therefore, the size of the RNA vector and the amount of vector that can fit into a lipid nano-carrier may be dose-limiting.

There remains a key opportunity to maximize the potential for therapeutic efficacy with RNA vaccines encoding peptide immunogens that have significantly improved biostability for sustained antigen expression and presentation and a smaller relative size to improve maximum potency and tolerability. A smaller relative size allows for encapsulation of a high copy number of immunogen-encoding vectors per nano-carrier for delivery to antigen presenting cells, maximizing antigen expression and presentation with less RNA and lipid. A small vector would also be practical to produce from synthetic RNA oligonucleotides, bypassing the need for cloning a DNA template. There is no vaccine currently available that meets all these needs, and it is anticipated that such a vaccine may not be possible based on the prior art. The possibility of producing the absolute maximum therapeutic response is critical for patients with hard-to-treat cancers, especially when success may be determined based on improving quality of life and longevity during the final months of life.

### SUMMARY OF THE INVENTION

Despite the success of mRNA-based vaccines, there has been an effort to identify technologies to improve biostability and pharmaceutical stability, and improve the efficacy of RNA vectors, mostly for the purposes of developing full length protein therapeutics that can be dosed repeatedly for chronic disease and that don't induce strong adverse innate immune responses. For this purpose, circularized RNA (circRNA) vector constructs have been shown to have improved biostability and pharmaceutical stability with long-lasting therapeutic protein/peptide translation, which occurs in a cap-independent manner.

However, mass production of chemically-defined long circRNA constructs from a DNA template that, like mRNA vectors, are generally several hundred or thousands of nucleotides long, is challenging because of the “run-off” feature of the T7 RNA polymerase and the consequent generation of difficult-to-remove heterogenous byproducts during the in vitro transcription of long mRNA. Traditional mRNA and long circRNA vectors have the same disadvantages due to large vector sizes and complex secondary structures that include long double-stranded RNA causing adverse side effects, the latter of which essentially are dose-limiting. Vector size limits the encapsulation density of immunogen-encoding RNA, and, in addition to the complex secondary structure including relatively long double-stranded RNA, is also directly responsible for innate immune activation that can cause vaccine reactogenicity and directly down-regulate antigen expression and presentation. This is not optimal for a vaccine technology. In contrast, mini oligonucleotide circRNA vaccine vectors that are chemically synthesized without a DNA template are attractive alternatives to leverage existing automated RNA oligonucleotide synthesis technology and manufacturing capabilities, chemically-defined RNA oligo synthesis and circRNA production, and the minimal functional elements in circRNA that make circRNA not only structurally minimal and compact, which enhance their loading capacity in nanocarriers, but also minimizes the possibility of potentially dose-limiting long double-stranded RNA in mini circRNA. It is unexpected and surprising that minimally-sized circRNA vectors with only a short internal ribosome entry site (IRES) sequence in the non-coding region can not only initiate and maintain efficient peptide translation, but also significantly prolong antigen expression and presentation and, consequently, enhance immunogenicity compared to conventional mRNA vaccines. Although previous implementations of circRNA have focused on not triggering innate immunity, mini-circRNA vectors are sufficiently self-adjuvanted, intrinsically activating endosomal and cytosolic immune sensors to provide innate immunity required for a powerful adaptive immune response. In practice, mini-circRNA vectors can be prepared by ligation with T4 ligase enzyme of one or multiple short oligonucleotides, which does not introduce extraneous RNA fragments containing immune-activating dsRNA, as compared to traditional permuted intron-exon (PIE) methods that have been currently used to synthesize long circRNA that intrinsically introduce extraneous RNA. The compact structure of mini-circRNA may explain why, paradoxically, these novel vectors demonstrate a multi-fold reduction in inflammatory chemokine activation compared to current state-of-the-art modified linear mRNA, that indicates potential for improved tolerability even despite enhanced antigen presentation and immunogenicity. The small size of mini-circRNA with a less complex secondary structure and no extraneous RNA fragments, such as regions of double stranded RNA, can likely minimize the potential for adverse inflammatory responses that limit the efficacy and safety of a candidate vaccine. This is similar to the incorporation of the modified nucleotide (e.g., pseudouridine) into traditional mRNA vaccines, which reduced intrinsic immunogenicity and thereby provided for superior translational capacity.

Accordingly, this disclosure provides synthetic minimal circular RNA (“mini-circRNA” or “circRNA”) that can be cost-effectively manufactured, and which can efficiently produce concatemer peptides by rolling circle translation (RCT) for subsequent peptide processing. When used as vaccines, the small size and biostability of mini-circRNA with a minimal non-coding region produces a multi-fold increase in immunogenicity due to the high encapsulation density of the vector and high copy number of immunogen-encoding RNA to antigen presenting cells, as well as efficient and sustained antigen expression over a prolonged duration due to high biostability of mini circRNA, and the concatemeric antigenic epitopes that are authentically processed by cellular proteases for optimal antigen presentation and T cell priming (similar effect as synthetic long peptide antigens). The small compact size of mini-circRNA was not previously a consideration by scientists designing RNA vaccines due to the need for long non-coding sequences to protect mRNA from degradation. However, the durability of circRNA results from the lack of end termini, which prevents exonuclease degradation and extends the lifespan of these molecules compared to linear RNA. This, unexpectedly, allows for the possibility of creating mini-circRNA vectors that maximize both vaccine potency and relative tolerability. Mini circRNAs are thus exceptionally well suited for use as vaccines, exponentially amplifying antigen translation via RCT and thereby eliciting/augmenting antigen-specific immunity for applications in, for example, preventing and treating cancer. In certain applications, mini-circRNA may also have advantages as vaccines for infectious disease.

It is an object of this invention to provide a mini circular RNA (circRNA) vaccine vector comprising 30 to 2200 nucleotides, constructed with 1 to 40 synthetic single-stranded oligonucleotide RNA sequences ligated together to form the mini circRNA vaccine vector. The circRNA comprises a coding region that is translated into a peptide or protein, and at least one non-coding region that is not translated. The non-coding region has at minimum an internal ribosome entry site (IRES), but other regulatory elements may also be included in the nucleotide sequences of the circRNA, such as a start codon, a stop codon, a KOZAK sequence, and any other regulatory element. In one embodiment of the invention, there is no stop codon immediately following the coding open reading frame. The mini circRNA vector with a minimum effective non-coding region is surprisingly effective and efficient at engaging ribosomes to initiate and maintain cap-independent translation. Since the non-coding region is restricted to a minimal size, most of the vector sequence encodes the immunogen(s). The small size allows a high encapsulation density of copies of immunogen-encoding RNA in each nano-carrier, which is then administered to a subject and delivered to antigen-presenting cells. A key benefit of the invention is the use of the lowest possible amount of non-coding RNA and fewest lipid nano-carriers per administered dose, as well as the reduced structural complexity of the vector, for example fewer regions of dsRNA, which can increase the maximum tolerable dose and prevent over-stimulation of innate immune sensors with potential adverse results. The combination of vector attributes is a breakthrough that results in specific immune T cell activation that paradoxically improves both immunogenicity and tolerability.

The coding region of the mini circRNA vaccine vector comprises nucleotide sequences encoding at least one immunogen of interest. In one embodiment, the immunogen induces cell-mediated immunity. In practicing the invention, cell-mediated immunity is induced in at least one cell type selected from the group consisting of immunogen-specific CD4⁺ T cells and immunogen-specific CD8⁺ killer T cells.

The sequence identity of the synthetic single-stranded oligonucleotide RNA sequences is determined by the overall nucleotide sequence of the immunogen or immunogens of interest and are designed, synthesized and combined in order to form the circRNA. In one embodiment, the synthetic single-stranded oligonucleotide RNA sequences are in the range of 40 to 150 nucleotides in length. The ratio of coding to non-coding sequences is a key feature of the invention, ranging from approximately 0.3 to 40. In one embodiment, the non-coding region is 50 to 300 nucleotides and the ratio of the nucleotide sequence length of the coding region to the non-coding region is in the range of 0.3 and 2 for a mini circRNA vaccine vector encoding a single immunogen. In another embodiment, the ratio is in the range of 1.5 and 10 for a mini circRNA vaccine vector encoding 2 to 5 peptide immunogens. In another embodiment, the ratio is in the range of 3 to 20 for a mini circRNA vaccine vector encoding 6 and 10 peptide immunogens. In yet another embodiment, the ratio is in the range of 6 to 40 for a mini circRNA vaccine vector encoding 11 and 20 peptide immunogens. In another embodiment, a non-coding region of 50 to 150 nucleotides is combined with the coding region and has a ratio in the range of 0.6 to 2, 3 to 10, 6 to 20, or 12 to 40. As will be seen in Examples of the invention, the ratio will be dependent upon the number of immunogens encoded by the vector, but it is a goal of the invention to have a high ratio of coding to non-coding sequences, and this high ratio provides greater efficacy. The non-coding region typically comprises 300 or fewer nucleotides, 150 or fewer nucleotides, 100 or fewer nucleotides, or fewer than 50 nucleotides. In one embodiment, the non-coding region consists of an internal ribosome entry site (IRES). The IRES may include but is not limited to an IRES in mRNA encoding LINE1, crTMV, Rbm3, or human c myc. Other IRES that may be used include but are not limited to KMI2 (98 nt); Apaf-1_-58/-3 (56 nt); BiP_-93_-1 (93 nt); c-IAP1_-81/-1 (81 nt); n-myc_-293_-207 (87 nt); n-myc_delta1-248 (79 nt); Rbm3_22 nt_module (22 nt); L-myc (52 nt); c-Myc (minimal IRES) (48 nt); LINE1-ORF2-138-86 (53 nt); crTMV_IRESmp75 (73 nt).

The coding region comprises nucleotide sequences encoding at least one immunogen. Immunogens may be any protein expressed by a cell of interest. Typically, the immunogen is a tumor-associated antigen. Also contemplated are nucleotide sequences encoding any of a tumor neoantigen, an oncoviral antigen and a testis cancer antigen. In one embodiment, the coding region comprises a nucleotide sequence encoding a plurality of peptide immunogens positioned consecutively with no peptide cleavage site or structural linker between the peptide immunogens. In this embodiment, the multiple immunogens are translated into a peptide concatemer which may then be processed into antigenic epitopes for MHC binding and antigen presentation. In another embodiment, the coding region encodes a plurality of peptide immunogens and the peptide immunogens are separated by linkers. These linkers may further encode peptide cleavage sites, structural peptide linkers and/or endoplasmic reticulum localization signal peptides.

One embodiment of the invention is a method of preparing a self-adjuvanted mini circRNA vaccine for a subject in need thereof, comprising 30 to 3300 nucleotides with at least one internal ribosome entry site (IRES) and encoding at least one immunogen. The circRNA is synthesized using the steps of:
providing one or more DNA splint(s),
   synthesizing linear single-stranded RNA oligonucleotides;
   hybridizing the DNA scaffold or splint(s) with at least two linear single-stranded RNA oligonucleotides to bring multiple oligonucleotides within local proximity of each other;
   ligating the single-stranded RNA oligonucleotides to form the mini circRNA vaccine vector;
   removing the DNA scaffold or splint(s); and
   purifying the mini circRNA vaccine vector.

After the circRNA is synthesized and purified, a mini circRNA vaccine vector is formulated. In one embodiment, the circRNA is solubilized in a pharmaceutically acceptable carrier, such as saline, buffered saline, or other biocompatible solution. In another embodiment, the mini circRNA vaccine vector is encapsulated in a nano-carrier selected from the group consisting of a liposome, an exosome, a polymeric nanoparticle, a protein nanoparticle, and a lipid nanoparticle. In another embodiment, the mini circRNA vaccine vector is modified with molecular targeting ligand to enhance targeted circRNA vaccine delivery.

It is an object of the invention to provide a method of treating cancer in a subject in need thereof, comprising the steps of identifying a cancer antigen expressed in cells of the cancer from which the subject suffers, synthesizing a mini circRNA vaccine vector wherein a coding region comprises nucleotide sequences encoding at least a portion of the cancer antigen and a non-coding region comprises at least one regulatory element. A therapeutically effective amount of the circRNA vaccine vector is administered to the subject to induce an immune response in the subject. The route of injection is typically by intravenous, intramuscular, intratumor, subcutaneous and/or intraperitoneal injection or infusion. It is an object of the invention to induce a maximal immune response. Thus, a further objective is to deliver a high copy number of circRNA molecules, each expressing a high ratio of coding to non-coding regions. The administering step may be repeated at intervals of 1 to 8 or more weeks.

The at least one immunogen may be any part or all of a tumor-specific antigen. In some embodiments, the immunogen is a mutant KRAS antigen, a melanoma tumor-specific antigen and/or an isocitrate dehydrogenase tumor-specific antigen. In another embodiment, the at least one immunogen is HLA-matched to the subject.

In one embodiment, the mini circRNA vaccine vector may be administered in combination with other cancer therapies, such as chemotherapy. In some embodiments, the immune response may be enhanced by coadministration with one or more immunotherapy agent such as a PD-1 inhibitor and/or PD-L1 inhibitor.

In another embodiment, the mini circRNA vaccine can be designed to express antigens derived from pathogenic microbes, such as viruses and bacteria. These circRNA vaccines may be used for the prophylaxis and therapy of the corresponding infectious diseases.

Other features and advantages of the present invention will be set forth in the description of invention that follows, and in part will be apparent from the description or may be learned by practice of the invention. The invention will be realized and attained by the compositions and methods particularly pointed out in the written description, Examples and claims hereof.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a schematic of synthesis of mini circRNAs. (Portion A) Linear RNAs were annealed to complementary DNA in the form of a circular DNA scaffold and then ligated using T4 RNA ligase 1 to generate a circRNA molecule. The DNA scaffold is removed with DNase treatment, followed by purification, leaving only the circRNA sequences. (Portion B) Mini circRNA is translated into concatemeric peptides via rolling circle translation (RCT), followed by proteolytic processing of the concatemeric peptides, resulting in prolonged antigen presentation. (Portion C) Nanocarriers deliver circRNA into lymphoid tissues (e.g., lymph nodes) and antigen-presenting cells to elicit antigen (Ag)-specific CD4+ and/or CD8+ T cell responses.

FIGS. 2A-B show small circRNA synthesis and peptide translation. (FIG. 2A) Agarose gel electrophoresis verified circRNA synthesis. (FIG. 2B) Western blot using denatured PAGE shows the expression of concatemeric FLAG by circRNA-FLAG in rabbit reticulocyte lysate (RRL) (24 h). The multiple large products indicate that they are likely translated via rolling circle translation.

FIGS. 3A-F illustrate the excellent biostability and pharmaceutical stability of small circRNA and circRNA LNPs. (FIG. 3A) Remaining circRNA, liRNA, and benchmark modified mRNA after storage in PBS at −20, 4, and 23° C. for up to 70 days (paired t-test). Data were quantified using ImageJ from agarose gel electrophoresis, as exemplified in (FIG. 3B). FIGS. 3C-3G illustrate circRNA stability studies in live DC2.4 cells using a reporter of DFHBI-1T-binding fluorogenic RNA aptamer, Broccoli. (FIG. 3C) Secondary structures of circBroccoli and Broccoli (predicted by RNAfold and Forna) indicated intact Broccoli structure in circBroccoli. (FIG. 3D) Confocal microscopy images (upper panel) (green: Broccoli-DFHBI-1T; blue: nuclei) and flow cytometry (lower panel) of DC2.4 cells treated with linear or circular Broccoli for 1-168 h, prior to adding DFHBI-1T before analysis. (FIG. 3E) DLS graphs showing the size distribution of blank and circRNA-loaded SM-102 LNPs. (FIG. 3F) Remaining circRNA after storage of circBroccoli LNPs in solutions at 4 or −20° C. (sucrose-supplemented) for 70 days, as quantified by flow cytometry of DC2.4 cells transfected with recovered circBroccoli LNPs (24 h). Statistical analysis for all data unless denoted otherwise: mean±s.e.m.; ns: non-significant; *p<0.05; **p<0.01; ***p<0.001; ****p<0.0001, one-way ANOVA with Bonferroni post-test.

FIGS. 4A-4E show that nanoparticle carriers promoted circRNA vaccine delivery. (FIG. 4A) Dynamic light scattering size distribution and a transmission emission microscopy image (inset) of liposomal circRNA (lipo-circRNA). (FIG. 4B) MFI of DC2.4 cells incubated with free or lipo-circRNA-Cy5, indicating efficient lipo-circRNA delivery. Inset: confocal microscopy showing lipo-circRNA delivery to DC2.4 cells (100 nM, 6 h). The circRNA not colocalized with Lysotracker indicates its endosome escape. (red: circRNA; green: endolysosome; blue: nucleus.) (FIG. 4C, FIG. 4D) IVIS imaging showing efficient circRNA-IR800 delivery (0.2 nmole, s.c. at foot pad) to draining popliteal lymph nodes (circled) in Balb/c mice. AUC: area under curve. (FIG. 5E) Intranodal cell subsets that took up liposomal or free circRNA-Cy5. Mig-DC: migratory DC.

FIGS. 5A-B show innate immunomodulation by circRNA nanovaccines (NVs). (FIG. 5A) circRNA NVs promoted the secretion of proinflammatory cytokines and IFN-β in DC2.4 cells. (FIG. 5B) siRNA silencing of Tlr7 or Rig-I reduced IFN-β response in DC2.4 cells treated with circRNA NVs (100 nM, 24 h).

FIGS. 6A-D show in vitro immunomodulation by small circRNA-SIINFEKL vaccine. (FIG. 6A) Flow cytometry mean fluorescence intensity (MFI) of antigen presentation (left) and priming of SIINFEKL-specific B3Z CD8⁺ T cell hybridoma (right) by circRNA-SIINFEKL-treated DC2.4 cells (100 nM circRNA, 1 μM CpG, 1 μM OVA; 24 h). (FIG. 6B) The secondary structure of crTMV-based circRNA-SIINFEKL (predicted by RNAfold and Forna). Blue box: IRES domain. (FIG. 6C) MFI of SIINFEKL presented on DC2.4 cells treated with circRNA mutants and benchmark modified mRNAOVA. (FIG. 6D) Relative to controls, circRNA-SIINFEKL-treated DC2.4 cells efficiently and durably primed B3Z T cell hybridoma. (C-D: 10 μg/mL RNA transfected by lipofectamine 3k.) FIGS. 7A-H show that low-dose circRNA-SIINFEKL NVs elicited potent and durable T cell responses in young adult mice (6 weeks) (FIGS. 7B-D) and immunosenescent aged mice (1 year) (FIGS. 7E-G). (FIG. 7A) Study design in mice (n=5). (FIG. 7B, FIG. 7C) Tetramer staining showed potent CD8⁺ T cell response by circRNA-SIINFEKL NVs that outperformed NVs of current benchmark 5-methoxyuridine-modified CleanCap® mRNAOVA and CpG+OVA. (FIG. 7D) circRNA NVs elicited antigen-specific T cell memory (day 70). (FIG. 7E) Kaplan Meier mouse survival showed that circRNA NVs-treated mice resisted the challenge of EG7.OVA cells (3×10⁵, s.c., day 71). *: relative to circRNA NVs. (FIG. 7F) In aged mice, circRNA NVs induced more antigen-specific CD8⁺ T cells than NVs of mRNAOVA or CpG+OVA (n=5) (day 21) (t-test). (FIG. 7G) Intracellular IFN-γ/TNF-α in CD8⁺ T cells from the as-immunized aged mice (day 35). (FIG. 7H) circRNA NVs protected aged mice from EG7.OVA cell challenge (3×10⁵, s.c., day 71). *: relative to circRNA NVs. (Vaccine in liposome: s.c. at tail base; 5 μg RNA, 2 nmol CpG, 20 μg OVA).

FIG. 8 shows that nanocarrier screening identified SM-102 LNPs to enable the most potent T cell responses by RNA vaccines. C57Bl/c mice (n=5) were immunized with different NVs of circRNA-SIINFEKL and modified mRNAOVA (5 μg RNA, s.c. injection at tail base; days 0, 14). SIINFEKL-specific PBMC CD8⁺ T cells were analyzed by tetramer staining on day 21.

FIGS. 9A-B showed by intracellular cytokine staining on day 21 that MHC-II-restricted circRNA-ISQ NVs not only induced antitumor effector CD4⁺ T cells (FIG. 9A) but also helper T cells to promote the CD8⁺ T cell responses induced by MHC-I-restricted circRNA-SIINFEKL NVs (FIG. 9B) in C57BL/6 mice (n=5). Single or dual circRNAs induced superior T cell responses to modified mRNA-OVA NVs. (s.c. injection; 5 μg RNA; day 0, 14)

FIG. 10 shows that circRNA SM102-LNPs had high tolerability. Luminex results of the serum cytokine/chemokine concentrations 12 h post vaccine administration in C57Bl/6 mice.

FIGS. 11-B shows a comparison of predicted secondary structures of RNA molecules. (FIG. 11A) Predicted secondary structures of traditional protein OVA-encoding mRNA, traditional SIINFEKL peptide-encoding mRNA, and SIINFEKL peptide-encoding mini circRNA. (FIG. 11B) Median fluorescence intensity (MFI) of SIINFEKL/MHC-1 presented on DC2.4 cells treated with the indicated RNA vaccines at a series of concentrations for 1-3 days. At all experiment conditions, SIINFEKL peptide-encoding mini circRNA outperformed OVA- or SIINFEKL-encoding mRNA modified with pseudouridine (Y) or 5 methyoxyl uridine (5 moU).

FIGS. 12A-G show results of low-dose circRNA NVs for combination tumor immunotherapy. (FIG. 12A, FIG. 12B) Study designs of Adpgk⁺ T cell analysis (A: n=5) and tumor immunotherapy (B: n=8) in C57BL/6 mice. (FIG. 12C-D) Tumor growth and body weights of EG7.OVA tumor-bearing mice treated with circRNA-SIINFEKL NVs vs. controls. (FIG. 12E) Tumor growth in TC-1 tumor-bearing mice treated with circRNA-E7 NVs vs. controls. (FIG. 12F) Neoantigen circRNA-Adpgk NVs induced dose-dependent T cell responses in C57BL/6 mice. (FIG. 12G) Tumor growth in syngeneic mice treated with circRNA-Agpgk NVs+αPD-1 vs. controls. mRNAOVA: modified with 5-methoxyuridine and CleanCap®. Vaccine: delivered by liposome, s.c. injected at tail base; 5 μg RNA, 2 nmol CpG, 20 μg protein or peptide antigens; antibodies: 200 μg, i.p. injection. *: relative to circRNA NVs.

FIGS. 13A-B show a tumor immune milieu analysis of MC38 tumor treated with circRNA-Agpgk NVs+αPD-1 vs. controls. (FIG. 13A) shows frequency of CD45+ cells as a percentage of total cells analyzed. (FIG. 13B) shows Adpgk-specific CD8+ T cells as a percentage of total cells analyzed and the CD8+/Treg ratio. mRNAOVA: modified with 5-methoxyuridine and CleanCap®. Vaccine: delivered by liposome, s.c. injected at tail base; 5 μg RNA, 2 nmol CpG, 20 μg protein or peptide antigens; antibodies: 200 μg, i.p. injection. *: relative to circRNA NVs.

FIG. 14 shows the effect of mini multi-circRNA nanovaccines (NVs) for ICB combination immunotherapy of melanoma. (Portion A) As a novel platform of mRNA vaccines, self-adjuvanted small multi-circRNA is comprised of 1) multivalent antigen (Ag)-encoding RNA and 2) peptide translation-initiating IRES and Kozak sequence. Via rolling circle translation (RCT), circRNA sustained the translation of concatemer antigens that are enzymatically processed and presented on APCs to elicit potent and durable immunity. (Portion B) Multi-circRNA nanovaccines are delivered to lymph nodes and APCs and elicit potent and durable T cell responses.

FIGS. 15A-D show the results of treatment with bivalent circRNA nanovaccines for combination melanoma immunotherapy. (FIG. 15A) Structures of circRNA-Trp2/gp100 nanovaccines based on SM-102 LNPs. (FIG. 15B) Intracellular IFN-γ/TNF-α staining of CD8+ T cells (day 21) from C57Bl/c mice immunized with circRNA nanovaccines or peptide nanovaccines (days 0, 14) (n=5). (FIG. 15C) circRNA elevated PD-1 expression on CD8+ T cells, thereby sensitizing PD-1 for immune checkpoint blockade (ICB). (FIG. 15D) B16F10 melanoma growth in C57BL/6 mice treated with circRNA nanovaccines+αPD-1 vs. controls. Vaccines: delivered by LNPs, s.c. injection at tail base; 5 μg RNA, 2 nmol CpG, 20 μg antigens. αPD-1: 200 μg, i.p. injection.

FIG. 16 shows a graphical illustration of tetramer staining data demonstrating that circRNA-RBD₄₄₀₋₄₅₉ elicited SARS-CoV-2 spike protein RBD epitope-specific CD8 T cell responses in mice. Vaccines: delivered by SM102-LNPs, s.c. injection at tail base; 5 μg RNA, 2 nmol CpG, 20 μg peptide antigens.

FIGS. 17A-B show manufacturing of a mini circRNA vaccine vector enabling a potent and durable immune response to two immunogens. (FIG. 17A) A simplified schematic drawing of the workflow for manufacturing a mini circRNA is shown. Single-stranded RNA oligonucleotides (oligos) are synthesized. A mixture of the RNA oligos and DNA splint(s) self-assembles and are ligated. The product is purified for formulation as a mini circRNA vaccine vector. (FIG. 17B) Many copies of the mini circRNA vaccine vectors are able to be packaged within nanocarriers, which are administered to a subject. For example, 185 copies of SIINFEKL-encoding mini circRNA (111 nucleotides) were loaded per SM102-LNP, in contrast to only 14 copies of OVA-encoding modified mRNA (1441 nucleotides) per such LNP. The nanocarriers are taken up by antigen presenting cells (APCs) and the mini circRNAs stimulate a robust immune response involving CD4+ and/or CD8+ T cells due to the multiple copy number. The activated CD4+ and/or CD8+ T cells in turn stimulate a potent and durable immune response via antigen-specific effector T cells and memory T cells.

FIGS. 18A-B show manufacturing of a mini circRNA vaccine vector enabling a potent and durable immune response to five or more immunogens. (FIG. 18A) A simplified schematic drawing of the workflow for manufacturing a mini circRNA is shown. Single-stranded RNA oligos are synthesized. The diagram indicates that at least five immunogens are encoded in a series of oligos. A mixture of the oligos self-assembles by annealing to a DNA scaffold or to DNA splints that bridge the oligos and form the desired structure. After the oligos are annealed to the scaffold or splints, the abutting ends of the oligos are ligated. The DNA is removed with DNase to enzymatically degrade the splints. The mini circRNA product is purified for formulation as a mini circRNA vaccine vector. (FIG. 18B) Many copies of the mini circRNA vaccine vectors are able to be packaged within nanocarriers, which are administered to a subject. The nanocarriers are taken up by antigen presenting cells (APCs) and the mini circRNAs stimulate a robust immune response involving CD4+ and/or CD8+ T cells due to the multiple copy number. The activated CD4+ and/or CD8+ T cells in turn stimulate a potent and durable immune response via antigen-specific effector T cells and memory T cells.

FIGS. 19A-C. The relative size of the immunogen encoding sequence to the non-coding region is a key element of the mini circRNA and contributes to the potent and durable immune response that the vaccine elicits. The size of a region refers to the number of RNA nucleotides (nts). (FIG. 19A) A mini circRNA vector having a single immunogen typically has a ratio of coding to non-coding nts between 0.33 and 2 and is above 0.67 in most embodiments. A vector having 2-5 immunogens typically has a ratio of coding to non-coding nts between 1.67 and 10 and is above 3.33 in most embodiments. A vector having 6-10 immunogens typically has a ratio of coding to non-coding nts between 3.33 and 20 and is above 6.67 in most embodiments. A vector having 11-20 immunogens typically has a ratio of coding to non-coding nts between 6.67 and 40 and is above 13.33 in most embodiments. (FIG. 19B) and (FIG. 19C) show traditional circular and linear RNA vectors, respectively. The non-coding regions are significantly larger than those of a mini circRNA vector, thus the ratio for a vector carrying the same payload of immunogens is significantly lower for the traditional vectors. (FIG. 19B) For a traditional circRNA encoding a single immunogen, the ratio of non-coding to coding regions is less than 0.3, and less than 0.15 in most instances. The ratio of non-coding to coding regions for 6-10 immunogens is less than 3 and is usually less than 0.8. The relative size of immunogen encoding sequence and non-coding region sequence in (FIG. 19C) traditional linear mRNA encoding a single immunogen the ratio of non-coding to coding regions is less than 0.3, and less than 0.1 in most instances. The ratio of non-coding to coding regions when comprising 6-10 immunogens is less than 3, and less than 0.4 in most instances.

FIGS. 20A-C show that equivalent amounts of RNA (i.e., equivalent numbers of RNA nucleotides) can be packaged in nanocarriers and delivered to APCs in the form of a mini circRNA vaccine vector, a traditional mRNA vaccine or a traditional circRNA. However, (FIG. 20A) the mini circRNA of the invention enables packaging and delivery of a greater number of copies of specific immunogen(s) of interest to APCs. A similar nanocarrier is able to carry fewer copies of (FIG. 20B) a traditional linear mRNA or (FIG. 20C) a traditional circRNA since these require inclusion of comparatively more non-coding nucleotides.

FIGS. 21A-B show a size comparison between a typical epitope-coding mini circRNA (FIG. 21A) and a traditional epitope-coding mRNA (FIG. 21B), with non-coding regions. The additional non-coding regulatory elements in red are minimal in the mini circRNA molecule compared to the traditional mRNA molecule. The bulk and steric hindrance of non-coding regions of the traditional mRNA take up space in carriers that can be filled by many more copies of the mini circRNA. In addition, the relatively long non-coding sequences in traditional mRNA enhanced the adverse reactogenicity of mRNA vaccines associated with immunostimulation by RNA and nucleotides and protein kinase K activation by long double-stranded RNA.

### DETAILED DESCRIPTION

The present invention provides synthetic oligonucleotide mini circRNAs that encode single or multivalent peptide antigens as vaccines. When formulated in a suitable carrier and administered to a subject, one or more peptide antigens encoded by the mini circRNA are efficiently produced as peptide concatemers by rolling circle translation (RCT) and processed into distinct epitopes for presentation in antigen presenting cells, thereby eliciting robust immune responses to target antigens. Due to high biostability (e.g., resistance to catabolism), high efficiency of ribosome binding to its compact structure, high encapsulation density, and less direct immunogenicity, antigen translational capacity and presentation with mini circRNA is substantially more robust and sustained compared to state-of-the-art mRNA peptide vaccines, resulting in significantly increased T cell activation. Paradoxically, the small relative size and limited two-dimensional structural complexity of mini circRNA vectors significantly reduces inflammatory chemokine activation which can portend improved tolerability, albeit while being sufficiently self-adjuvanted to produce a robust adaptive immune response. This is not dissimilar from the incorporation of the modified nucleotide pseudouridine into traditional mRNA vaccines, which reduced intrinsic immunogenicity and improved biostability with enhanced translational capacity.

As used herein, the circular RNA of the invention may also be referred to interchangeably as “mini circRNA”, “mini-circRNA” or “small circRNA”. Circular RNA is a type of single-stranded RNA which, unlike linear RNA, forms a covalently closed continuous loop. Natural circRNAs are products of precursor mRNA (pre-mRNA) back-splicing in eukaryotes that have various biological functions as noncoding or coding RNAs. In circRNA, the 3′ and 5′ ends normally present in an RNA molecule are joined together. Because circRNA does not have 5′ or 3′ ends, it is resistant to exonuclease-mediated degradation and is presumably more stable than most linear RNA in cells. Naturally occurring forms of circRNA exhibit a wide range of sizes, ranging from about 250-4000 nucleotides. In contrast, the synthetic mini circRNAs disclosed herein generally have a size ranging from approximately 30 to 1000 nucleotides (nts). In some embodiments, the size range is from approximately 80 to 2000 nts. In other embodiments, the size range is up to approximately 3300 nts. While there is some overlap in size with naturally occurring forms of circRNA, the mini circRNAs of the invention are synthesized to include coding regions which encode for one or more immunogens and/or one or more copies of the same immunogen. Thus, the mini circRNAs of the invention include noncoding regions that are minimized by comparison to naturally occurring circRNAs.

As used herein, the term “DNA splint” refers to a short DNA oligonucleotide used as a temporary bridge between two RNA oligonucleotides. The DNA splint is designed to be complimentary to nucleotide sequences at the ends of RNA oligos to be joined to form the mini circRNA. Splint ligation of RNA oligonucleotides, whereby specific RNA oligonucleotides are ligated together, can be carried out using T4 RNA ligase or T4 DNA ligase and bridging DNA oligonucleotide complementary to the RNAs. While T4 RNA ligase may be preferred in some embodiments of the invention, the method takes advantage of the property of T4 DNA ligase to join RNA molecules when they are in an RNA:DNA hybrid. Thus, a 5′ portion of DNA splint is complimentary to a 3′ portion of a first RNA oligo and a 5′ portion of a second RNA oligo. When the RNA oligos are annealed to the DNA splint, a T4 ligase enzymatically joins the ends of the RNA oligos and forms a circular RNA molecule. Subsequent treatment with DNase removes the DNA splint, leaving only the circularized RNA molecule. Alternatively, a circular DNA molecule may be synthesized and used as a scaffold for hybridizing with a series of complementary RNA oligonucleotides. Incubation with a T4 ligase connects the ends of the RNA oligos, forming a circular RNA:DNA hybrid, which is then treated with DNase to remove the DNA scaffold.

As used herein, “antigen” refers to a substance that binds to a component of the immune system (e.g., lymphocytes and their receptors). “Immunogen” refers to a subset of antigens that induce an immune response in the body, especially the production of antibodies or activation of T cells. Immunogens are often proteins or protein subunits, but immune responses can also be induced against lipids and nucleic acids. “Epitope”, also called antigenic determinant, refers to a portion of an antigen or immunogen, that is capable of binding to the component of the immune system to stimulate an immune response. An epitope is the part of an antigen or immunogen that is recognized by the immune system, specifically by antibodies, B cells, and/or T cells. The epitope is the specific piece of the antigen to which an antibody binds or that is presented to T cells. Herein, “antigen” and “epitope” may be used interchangeably and may be replaced by “immunogen”. An “antigenic region” generally refers to an amino acid sequence that comprises at least one epitope/antigen/immunogen.

As used herein, the terms “noncoding region” and “non-translated region” are used to describe a region of nucleotide sequences that are not translated into a peptide or protein. The terms “non-coding region”, “non-translated region” and “non-coding nucleotides” may be used interchangeably. Furthermore, the ratio of coding to non-coding region sequence length is a feature of the invention. More specifically, the number of nucleotides of the coding regions to the number of nucleotides of non-coding regions is very high compared to conventional RNA vaccines. Most RNA vaccines encoding peptide epitopes have a coding region of 50 to 1000 nucleotides of RNA and a non-coding region of about 500 to 2000 nucleotides, thus the ratio of coding to non-coding is in the range of less than 0.1 to 2. The circRNA of the invention generally has coding region of 50 to 2000 nucleotides, and more commonly 50 to 500 nucleotides, and a non-coding region of 50 to 300 nucleotides, thus the coding to non-coding ratio for the circRNA of the invention is in the range of 1 to 20. In some embodiments, the non-coding region is less than 100, and the ratio will be at the high end of the range. The high ratio coding RNA to RNA ensures that a very high density of immunogen delivered to each antigen presenting cell. The expected ratio is dependent on the number of immunogens in the coding sequence. CD4 epitopes typically have about 10 to 20 amino acids (30 to 60 nucleotides) and CD8 epitopes have about 6 to 10 amino acids (18 to 30 nucleotides). The size of each immunogen may be larger than the targeted epitope, and some immunogens may be intended to encompass multiple potential epitopes depending on the HLA genetics of the individual patient. However, in most cases immunogens are not expected to be greater than 30 amino acids (90 nucleotides). The coding region can also include peptide cleavage sites and/or peptide linkers; however, these sequences do not encode the immunogen and, therefore, are not included in calculating the coding to non-coding ratio.

As used herein, the terms “regulatory element” and “control element” refer to nucleotide sequences in non-coding regions that regulate translation of a coding region. It is important to note that regulatory elements are encoded in the nucleotide sequence but are not translated into a peptide or protein product of the nucleotide sequence. Examples of regulatory elements used in the invention include IRES, Kozak sequence, translation initiation site, stop codon, start codon, and other regulatory elements that are known in the art.

The term “Kozak consensus sequence” (used interchangeably with Kozak consensus or Kozak sequence) refers to a nucleic acid motif that functions as the protein translation initiation site in most eukaryotic mRNA transcripts. The Kozak sequence was initially described by Kozak (1987, Nucleic Acids Res., vol. 15, pp. 8125-8148). The Kozak sequence on an mRNA molecule is recognized by the ribosome as the translational start site. In some embodiments, the Kozak sequence is (gcc)gccRccAUGG, wherein R=G or A.

The term “internal ribosome entry site (IRES)” refers to an RNA element that allows for translation initiation in a cap-independent manner, as part of the greater process of protein synthesis. IRES sequences can only be identified experimentally. There is no consensus sequence or RNA structure that defines an IRES. IRESs cannot be predicted bioinformatically. In fact, both short unstructured sequences and long structured 5′ untranslated regions (UTRs) have been demonstrated to have IRES activity.

As used herein, the terms “linkers” and “spacers” are used interchangeably to refer to a short string of nucleotides that may be coding or non-coding. Linkers or spaces are typically only a few nucleotides in length, and usually less than about 12 nucleotides. Linkers or spacers can be used to separate the immunogens in a coding region. In this case, the linkers or spaces are likely to comprise multiples of 3 nts to keep the immunogen nucleotide sequences “in frame” for translation into protein products. Linkers or spacers may allow a small degree of separation between the immunogens, or they may encode a protease cleavage site or sites that allow cleavage of the immunogen products.

One embodiment of the invention is a method of preparing a self-adjuvanted mini circular RNA (circRNA) vaccine for a subject in need thereof, comprising 30 to 3300 nucleotides with at least one internal ribosome entry site (IRES) and encoding at least one immunogen. An exemplary circRNA is synthesized using the steps of:
synthesizing one or more DNA splint(s), wherein each half of a DNA splint is complementary to one of the RNA termini that are designed to be ligated into the form of a circRNA, thereby bringing these RNA termini into close proximity;
synthesizing linear single-stranded RNA oligonucleotides;
annealing or hybridizing the DNA splints with one or more linear single-stranded RNA oligonucleotides to bring the designed pairs of 5′ and 3′ ends of the same linear single stranded RNA oligonucleotides or the 5′ and 3′ ends of different linear single-stranded RNA oligonucleotides within close proximity of each other;
ligating the 5′ and 3′ ends of the one or more single-stranded RNA oligonucleotides to form the mini circRNA;
removing the DNA splints and unligated linear single-stranded RNA oligonucleotide;
purifying the mini circRNA; and
concentrating, lyophilizing, or drying the mini circRNA as needed.

After the circRNA is synthesized and purified, it may be stored for future use, or it may be formulated, for example, as a vaccine, i.e., a mini circRNA vaccine vector, and stored or administered to a subject. In one embodiment, the circRNA is solubilized in a pharmaceutically acceptable carrier, such as saline, buffered saline, or other biocompatible solution. In another embodiment, the mini circRNA vaccine vector is encapsulated in a nano-carrier selected from the group consisting of a liposome, an exosome, a nanoparticle and a lipid nanoparticle.

In one embodiment, the invention is a platform for rapidly designing and producing vaccines. The mini-circRNAs are advantageously designed in a modular manner so that it is possible, for example, to remove antigen-encoding sequences from a circRNA and insert new antigen-encoding sequences while retaining control sequences. This eliminates the need to redesign all aspects of each new circ-RNA and permits efficient production. Alternatively, one or more control sequences may be removed and replaced by different control sequences, e.g., to tailor the vaccine to different host recipients (e.g., to various types of mammals, to hosts of different ages, etc.). The modularity of circRNA structures permits them to be widely applicable for the development of small mRNA vaccines and therapeutics of all types.

Compared to conventional linear mRNA, mini-circRNAs have several advantageous features, including but not limited to:
1) induces robust antigen-specific immunogenicity that results in potent and long-lasting T cell responses by CD8⁺ cytotoxic T cells, CD4⁺ effector T cells and CD4⁺ helper T cells;
2) intrinsic immune adjuvanting (they are self-adjuvanted) by activating pattern recognition receptors to induce proinflammatory cytokines, without eliciting overly strong immunotoxic innate immune responses; there is no requirement for the use of modified nucleotides, such as pseudouridine, that have been otherwise been used to improve the tolerability of many linear mRNA vaccines;
3) chemically-defined manufacturing of circRNA using a semi-automated production method; and
4) the modular circRNA system allows for rapid customization and modification, which is particularly critical for personalized medicine and quickly adaption for application in emerging diseases.

Furthermore, a circRNA can express single or multivalent peptide concatemers that undergo intracellular antigen processing and presentation (similar to long synthetic peptides), resulting in potent and durable antigen-specific immune responses.

It is an object of this invention to provide a mini circRNA vaccine vector. In one embodiment, the mini circRNA vector comprises 30 to 3300 nucleotides, which are constructed from 1 to 40 synthetic single-stranded oligonucleotide RNA sequences ligated together to form the mini circRNA vector. In another embodiment, the mini circRNA is constructed from 80 to 2000 nucleotides, and the number of synthetic RNA oligos needed is in the range of 2 to 40. In another embodiment, the mini circRNA is constructed from 30 to 450 nucleotides, and the number of synthetic RNA oligos needed is in the range of 2 to 12, 2 to 11, 2 to 10 or 2 to 9. In another embodiment, the mini circRNA is 30 to 200 nucleotides, and is constructed from 2 to 5, 2 to 4, 2 to 3, or as few as 2 synthetic RNA oligos.

The circRNA comprises a coding region that is translated into one or more peptides or a protein. The non-coding region has, at minimum, an internal ribosome entry site (IRES), but other regulatory elements may also be included in the nucleotide sequence of the non-coding region, such as a start codon, a stop codon, a Kozak sequence, and any other regulatory elements. In one embodiment of the invention, there is no stop codon at the 3′-end of the open reading frame of the coding region. The non-coding region may be restricted to a single region of the circRNA, or it may be divided between two or more regions of the overall circRNA.

In one embodiment, the mini circular RNA vaccine vector has 80 to 450 nucleotides, including a coding region with a nucleotide sequence that encodes a single peptide immunogen or multiple peptide immunogens and non-coding regions that are, as defined below, not translated to produce peptides, and the relative sequence length or ratio of the coding region to the non-coding region is between 0.3 and 2, while in another embodiment the ratio of coding region to the non-coding region is between 0.6 and 2.

In another embodiment, the mini circular RNA vaccine vector has 120 to 1050 nucleotides, including a coding region having a nucleotide sequence encoding between 2 and 5 peptide immunogens, and the relative sequence length or ratio of the coding region to the non-coding region is between 1.5 and 10, while in another embodiment the ratio of the coding region to the non-coding region is between 3 and 10.

In another embodiment, the mini circular RNA vaccine vector has 250 to 1800 nucleotides, including a coding region having a nucleotide sequence that encodes between 6 and 10 peptide immunogens, and the relative sequence length of the coding region to the non-coding region is between 3 and 20. In some embodiments the ratio of the coding region to the non-coding region is between 6 and 20.

In yet another embodiment, the mini circular RNA vaccine vector has 450 to 3300 nucleotides, including a coding region comprising a nucleotide sequence encoding between 11 and 20 peptide immunogens, and the relative sequence length of the coding region to the non-coding region is between 6 and 40. In some embodiments the ratio of the coding region to the non-coding region is between 12 and 40.

In some embodiments, the mini circular RNA vaccine vector has 85 to 3300 nucleotides, including a coding region comprising a nucleotide sequence that encodes between 1 and 20 peptide immunogens with a non-coding region of between 50 and 150 nucleotides. The ratio is dependent upon the length of the coding sequence and the length of the non-coding sequence. The ratio of the length of the coding sequence to the non-coding sequence determines the encapsulation density of the circRNA in nanocarriers, and thereby the copy number of coding RNA per nanocarrier delivered to cells (e.g., antigen presenting cells). This can also be increased by a small circRNA size and large copy number of a peptide encoded by one circRNA. In other words, increasing the ratio of coding/noncoding means the same as expanding the coding region (increase the vaccine valency in one circRNA). It was unexpected and surprising that the compact size of the non-coding region of mini-circRNA vectors is sufficient to initiate and maintain ribosome translation, and even more so that the small relative size of the non-coding region could increase efficacy by increasing encapsulation and vector delivery in nanocarriers. As will be seen in Examples of the invention, the ratio will be dependent upon the sizes of the coding to non-coding regions, but it is a goal of the invention to have a high ratio of coding to non-coding sequences, and this high ratio allows greater efficacy. The non-coding region typically comprises 300 or fewer nucleotides, 150 or fewer nucleotides, 100 or fewer nucleotides, or fewer than 50 nucleotides. Thus, when the mini-circRNA coding region encodes a single peptide immunogen, the relative sequence length of the coding region to the non-coding region may be 0.3 to 2, or it may be 0.6 to 2, depending upon the precise lengths of each region. In another example, if the coding region encodes between 2 and 5 peptide immunogens, the relative sequence length of the coding region to the non-coding region may be 1.5 to 10, or it may be 3 to 10. If the coding region encodes between 6 and 10 peptide immunogens, the relative sequence length of the coding region to the non-coding region is between 3 and 20, or it could fall between 6 and 20. If the coding region encodes between 11 and 20 peptide immunogens, the relative sequence length of the coding region to the non-coding region is to fall between 6 and 40, or it could be between 12 and 40. One of skill in the art will recognize that the precise ratio for any particular embodiment will be calculated in this manner. The coding region of the mini circRNA vaccine vector comprises nucleotide sequences encoding at least one immunogen of interest. In one embodiment, the immunogen induces cell-mediated immunity. In practicing the invention, cell-mediated immunity is induced in at least one cell type selected from the group consisting of immunogen-specific CD4⁺ T cells and immunogen-specific CD8⁺ killer T cells.

The sequence identity of the synthetic single-stranded oligonucleotide RNA sequences is determined by the overall nucleotide sequence of the immunogen or immunogens of interest and are designed, synthesized, and combined in order to form the circRNA. In one embodiment, the synthetic single-stranded oligonucleotide RNA sequences are in the range of 40 to 150 nucleotides in length. In another embodiment, the synthetic single-stranded oligonucleotide RNA sequences are in the range of 40 to 80 nucleotides in length. Smaller oligonucleotides may be simpler and less expensive to produce; however, a larger number of oligonucleotides and ligations will be required to produce a given vector. In one embodiment, a mini-circRNA vector comprising 80 to 2000 nucleotides, is constructed with 2 to 40 synthetic single-stranded oligonucleotide RNA sequences ligated together to form the mini circRNA vaccine vector, comprising a non-coding region comprising an internal ribosome entry site (IRES), a coding region comprising nucleotide sequences encoding at least one immunogen, and optionally comprising nucleotide sequences encoding one or more linkers or spacers. In another embodiment, a mini-circRNA vector comprising 80 to 1000 nucleotides, is constructed with 2 to 20 synthetic single-stranded oligonucleotide RNA sequences ligated together to form the mini circRNA vaccine vector. In another embodiment, a mini-circRNA vector comprising 80 to 500 nucleotides, is constructed with 2 to 10 synthetic single-stranded oligonucleotide RNA sequences ligated together to form the mini circRNA vaccine vector. In yet another embodiment, a single synthetic single-stranded oligonucleotide RNA sequence representing the non-coding region may be ligated to one or more synthetic single-stranded oligonucleotide RNA sequences encoding between 1 and 20 immunogens and/or multiple repeats of one or more immunogen. In yet another embodiment, the same single synthetic single-stranded oligonucleotide RNA sequence for the non-coding region may be combined with different coding oligonucleotides to produce mini-circRNA vaccine vectors encoding different immunogens but with the same regulatory elements.

In one embodiment, the non-coding region consists of an IRES. In general, the IRES is less than 300 nucleotides. The IRES may be an IRES is selected from the group consisting of LINE1, crTMV, Rbm3 and human c-myc; in other words, the IRES of a human c-myc mRNA, the IRES of a Rbm3 mRNA, etc.

The coding region comprises nucleotide sequences encoding at least one immunogen. Immunogens may be any whole or parts of peptides or proteins to be expressed by a cell of interest that is the target of the vector. In the primary embodiment, immunogens are peptide immunogens that represent potential antigens to be expressed in antigen presenting cells or splenic cells. In another embodiment, the immunogen may be a therapeutic protein of interest. For embodiments for treating cancer, the immunogens are commonly cancer-associated antigens, cancer neoantigens, oncoviral antigens, cancer testis antigens, etc. Also contemplated are nucleotide sequences encoding any of a tumor neoantigen, an oncoviral antigen and a testis cancer antigen or a combination thereof. In one embodiment, the coding region comprises a nucleotide sequence encoding a plurality of peptide immunogens positioned consecutively with no peptide cleavage site or structural linker between the peptide immunogens. In this embodiment, the multiple immunogens are translated into a peptide concatemer. The peptide concatemer may need to be processed intracellularly into peptide epitopes to be presented as antigens. In another embodiment, the coding region encodes a plurality of peptide immunogens and the peptide immunogens are separated by linkers. These linkers may further encode peptide cleavage sites or structural peptide linkers. Peptide cleavage sites may determine the site of antigen processing as opposed to allowing for random processing of peptide concatemers in vectors with peptide cleavage sites.

One embodiment of the invention is a method of preparing a self-adjuvanted mini circular RNA (circRNA) vaccine for a subject in need thereof, comprising 80 to 2000 nucleotides with at least one internal ribosome entry site (IRES) and encoding at least one immunogen. The circRNA is synthesized using the steps of:
providing one or more DNA splint(s);
synthesizing linear single-stranded RNA oligonucleotides;
hybridizing the DNA splints with at least two linear single-stranded RNA oligonucleotides to bring multiple oligonucleotides within local proximity of each other;
ligating the single-stranded RNA oligonucleotides to form the mini circRNA vaccine;
removing the DNA splints; and
purifying the mini circRNA vaccine.

After the circRNA is synthesized and purified, a mini circRNA vaccine vector is formulated. In one embodiment, the circRNA is solubilized in a pharmaceutically acceptable carrier, such as saline, buffered saline, or other biocompatible solution. In another embodiment, the mini circRNA vaccine vector is encapsulated in a nano-carrier selected from the group consisting of a liposome, an exosome, a nanoparticle and a lipid nanoparticle.

It is an object of the invention to provide a method of treating cancer in a subject in need thereof, comprising the steps of identifying a cancer antigen expressed by cells of the cancer from which the subject suffers, synthesizing a mini circular RNA (circRNA) vaccine vector wherein a coding region comprises nucleotide sequences encoding at least a portion of the cancer antigen and a non-coding region comprises at least one regulatory element. A therapeutically effective amount of the circRNA vaccine vector is administered to the subject induce an immune response in the subject. The route of injection is typically by intravenous, intramuscular, intratumoral, subcutaneous, intradermal, intracranial, and/or intraperitoneal injection or infusion. It is an object of the invention to induce a maximal immune response. Thus, a further objective is to deliver a high copy number of circRNA molecules, each expressing a high ratio of coding to non-coding regions. The administering step may be repeated at intervals of 1 to 8 or more days or weeks.

The at least one immunogen may be any part or all of a tumor-specific antigen. In some embodiments, the immunogen is a mutant KRAS antigen, a melanoma tumor-specific antigen and/or an isocitrate dehydrogenase tumor-specific antigen. In another embodiment, the at least one immunogen is HLA-matched to the subject.

In one embodiment, the mini circRNA vaccine vector may be used in combination with other cancer therapies, such as surgery, chemotherapy, radiotherapy, and any other immunotherapy that a practitioner may deem suitable. In some embodiments, the immune response may be enhanced by coadministration with one or more immunotherapy agent such as a PD-1 inhibitor and/or PD-L1 inhibitor.

In one embodiment, the mini circRNA vaccine vector may encode viral antigenic peptides or proteins. Examples of these viral antigens include spike protein or its subunit epitopes from SARS-COV and SARS-CoV-2, extracellular domain of matrix 2 (M2e) or hemagglutinin from influenza, gp120 from human immunodeficiency virus (HIV), and E7 proteins from human papillomavirus (HPV). These circRNA vaccines can be used prophylactically to prevent the recipients from being infected with the corresponding pathogens, and can also be used therapeutically, alone or in combination with other therapeutics, to treat pre-existing infections.

### Elements of the Mini CircRNA

The mini circRNAs described herein comprise several elements. Among these, two are required: 1) at least one (and usually only one) internal ribosome entry site (IRES) that initiates protein translation in a cap-independent manner; and 2) an epitope-encoding region, i.e., RNA nts that code for translation of at least one immunogen. Other elements that are optional include but are not limited to: 3) at least one (and usually only one) Kozak sequence which initiates protein translation in eukaryotic systems; 4) one or more stop codons to signal the end of a translated epitope-encoding region; and, if multiple epitopes are encoded as a single transcript, 5) at least one sequence that encodes a protease cleavage site, e.g., between each epitope for predetermined epitope processing or other peptide linkers. In some embodiments a Kozak sequence can be used in place of an IRES. Each of these elements will be described more fully in subsequent passages.

In some aspects, the circRNAs described herein comprise three sections: 1) an IRES that initiates protein translation in a cap-independent manner, 2) a Kozak sequence which initiates protein translation in eukaryotic systems; and 3) an epitope-coding region.

In some aspects, the circRNAs described herein comprise four sections: 1) an IRES that initiates protein translation in a cap-independent manner, 2) a Kozak sequence which initiates protein translation in eukaryotic systems; 3) an epitope-coding region; and 4) one or more stop codons.

In other aspects, the circRNAs comprise minimally five sections: 1) an IRES that initiates protein translation in a cap-independent manner 2) a Kozak sequence that initiates protein translation in eukaryotic systems; 3) an epitope-coding region that ends with 4) a stop codon; and 5) at least one sequence that encodes a protease cleavage site. Protease cleavage sites include but are not limited to an AYY linker, a GPGPG linker, 2A self-cleavage peptides, endoplasmic reticulum-homing signal peptides, aspartic protease cleavage sites, cysteine protease cleavage sites, metalloprotease cleavage sites and serine protease cleavage sites.

A stop codon is typically encoded in an RNA molecule as UAG, UAA, or UGA. In an important note, the circRNAs would often intentionally not contain a stop codon or protease cleavage sites to optimize the vector for rolling circle translation, wherein ribosomes either remain attached to the circular vector for multiple cycles or quickly reengage the compact mini-circRNA to continuously translate encoded peptides as concatemers, which would then be naturally processed intracellularly into epitopes without predetermined cleavage sites.

The present modular or cassette approach to the mini circRNA design advantageously permits the elements to be easily swapped or replaced. This is especially true for mini-circRNAs prepared with synthetic single-stranded oligonucleotide RNAs, as automated synthesizers can be used to rapidly produce any customized synthetic single-stranded oligonucleotide RNA sequence to modify the regulatory elements and/or target immunogens. For example, the control elements can be changed for optimal translation in different hosts or different immunogens. In addition to swapping regulatory elements, the immunogens can easily be swapped to produce specific vaccines targeting different immunogens. In particular, it is advantageous to be able to remove a particular epitope-encoding region and replace it with a region that encodes at least one different epitope of interest, thereby producing at least one different epitope in the host. In one embodiment, a single synthetic single-stranded oligonucleotide RNA would be prepared for the non-coding region, which could be employed with any number of synthetic RNA oligonucleotides encoding one or more specific immunogens. The same non-coding oligonucleotide RNA could then be combined with different immunogen-encoding oligonucleotide RNAs targeting a different immunogen or a different set of immunogens.

It is important to note that the number of nucleotides in the domains of the antigen-coding region and, if included, the Kozak sequence and/or stop codons, is altogether multiples of 3 to prevent frameshift errors after the first round of translation during rolling circle translation.

### Encoded Immunogen/Antigens/Epitope(s)

The mini-circRNAs of this disclosure comprise at least one (i.e., one or more) ribonucleotide sequences which encode an antigenic region, i.e., a region or segment of a peptide or protein (or other antigenic molecule) that comprises at least one potential epitope. When the antigenic region is translated in a subject to whom the mini-circRNAs are administered, at least one epitope elicits an immune response in the subject.

In some aspects, a single immunogen or epitope may be encoded. However, generally a mini circRNA comprises nucleotide sequences that encode multiple antigens/epitopes, i.e., generally they encode multivalent peptide antigens. Immunogens may be peptide antigens that require intracellular processing to yield epitopes, or immunogens may be encoded as individual epitopes that do not require processing to be presented to T cells. Generally, only individual epitopes would be separated by specific peptide cleavage sites.

In some aspects, a plurality of peptide immunogens are encoded in tandem and at least two, or possibly all, of the immunogens, usually individual epitopes, are separated by a peptide sequence that is a protease cleavage sequence. Thus, an RNA sequence is translated into a polypeptide in a host, and the polypeptide is cleaved into individual antigens/epitopes in vivo by endogenous proteases at predetermined cleavage sites. In other aspects, a plurality of peptide immunogens or epitopes encoded in tandem are not separated by a peptide cleavage site, and the polypeptide is cleaved in vivo by endogenous proteases without using predetermined cleavage sites.

The type and number of antigens/epitopes encoded may vary widely. For example, they may all be based on or originate from a single infectious agent such as a virus, bacterium, etc. As such, the individual epitopes may be from different sequences within a single protein or from different sequences of various different proteins of the infectious agent (e.g., the spike protein of SARS-CoV2). Alternatively, the epitopes may be from a combination of several different infectious agents (e.g., akin to diphtheria, pertussis, and tetanus (DPT) vaccines); or from different strains of a single infectious agent (e.g., akin to influenza vaccines). Further, combinations of these types of epitopes may be included in one type of circRNA, i.e., epitopes from multiple infectious agents may be included and multiple different antigens from each of the infectious agents may be included.

Notably, the epitopes need not originate from infectious agents. Cancer antigenic sequences may also be encoded as well as cancer-associated antigens, cancer neoantigens, oncoviral antigens, testis cancer antigens, self-antigens, allergenic antigens and so on.

In general, from about 1 to about 50 individual (separate) epitopes are encoded in a single circRNA, regardless of the origin of each. For example, about 2, 4, 6, 8, 10, 12, 14, 16, 18, 20, 22, 24, 26, 28, 30, 32, 34, 36, 38, 40, 42, 44, 46, 48 or 50 individual epitopes may be encoded. However, generally 10 or fewer individual epitopes are encoded in a single circRNA, such as about 1, 2, 3, 4, 5, 6, 7, 8, 9 or 10 epitopes. If multiple epitopes are present, the epitopes may each be different (have a different amino acid sequence) and/or some or all may be repeated. Each epitope is generally from about 5 to about 50 amino acids in length, e.g., from about 5, 6, 7, 8, 9, 10 and up to about 50 amino acids, including all whole integers between 10 and 50. Usually, the epitopes comprise about 7 to 30, such as about 8 or 9, or 15-20 7, 8, 9, 10, 11, 12, 13, 14, 15, 16, 17, 18, 19, 20, 21, 22, 23, 24, 25, 26, 27, 28, 29 or 30 amino acids, such as 8 or 9 amino acids, or from about 15-20 amino acids.

### Translation Initiation Sequence

The mini-circRNAs of this disclosure comprise a translation initiation sequence such as a Kozak sequence and IRES, or the combination of both or multiple of them. A translation initiation sequence is a ribonucleotide sequence that initiates protein translation in eukaryotic systems. The translation initiation sequence may comprise any eukaryotic start codon such as AUG, CUG, GUG, UUG, ACG, AUC, AUU, AAG, AUA, or AGG. In some aspects, the eukaryotic start codon is AUG. In other aspects, translation begins at an alternative translation initiation sequence, e.g., translation initiation sequence other than AUG codon, under selective conditions, e.g., stress induced conditions. As a non-limiting example, the translation of the circRNA may begin at an alternative translation initiation sequence, such as ACG, CTG/CUG or GTG/GUG. As yet another non-limiting example, the circRNA may begin translation at a repeat-associated non-AUG (RAN) sequence, such as an alternative translation initiation sequence that includes short stretches of repetitive RNA, e.g., CGG, GGGGCC, CAG, CTG.

In certain aspects, the translation initiation sequence may be or comprise a Kozak sequence or a functionally equivalent sequence. The Kozak sequence comprises an AUG start codon, immediately followed by a highly conserved G nucleotide: AUGG. In particular, a Kozak sequence may be identified by (gcc)gccRccAUGG (SEQ ID NO: 01), as follows: (i) lower case letters denote the most common base at a position where the base can nevertheless vary; (ii) upper case letters indicate highly conserved bases (e.g., AUGG); (iii) ‘R’ indicates a purine (adenine or guanine); (iv) the sequence in brackets ((gcc)) is of uncertain significance; (v) the underlined AUG base triplet represents the start codon.

In some aspects, the Kozak sequence is GCCACCAUG. In some aspects, the number of nucleotides in the Kozak sequences that are incorporated into the subject circRNAs are multiples of three, e.g., when stop codons are not present. Therefore, classic and exemplary Kozak sequences described herein may be altered to meet this requirement, e.g., by deletion or addition or one or more nucleotides to generate a suitable total number of nucleotides, as long as proper functioning of the sequence is retained.

In some embodiments, a translation initiation sequence can function as a regulatory element.

### IRES Elements

In some embodiments, the circRNAs described herein comprise an IRES element. A suitable IRES element to include comprises an RNA sequence capable of engaging a eukaryotic ribosome. In some embodiments, the IRES element is at least about 5 to 500 nt in length, e.g., at least about 8, 9, 10, 15, 20, 25, 30, 40, 50, 100, 200, 250, 350, 400, 450 or 500 nts in length. The IRES element maybe derived from the DNA or RNA of an organism including, but not limited to, a virus, a mammal, and an insect (e.g., Drosophila). Viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_SNCR, CrPV_IGR, crTMV_IREScp, crTMV_IRESmp75, crTMV_IRESmp228, crTMV_IREScp, crTMV_IREScp, CSFV, CVB3, DCV IGR, EMCV-R, EoPV SNTR, ERAV_245-961, ERBV_162-920, EV71_1-748, FeLV-Notch2, FMDV_type_C, GBV-A, GBV-B, GBV-C, gypsy_env, gypsyD5, gypsyD2, HAV_HM175, HCV_type_1a, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1_-302_to_-202, LINE-1_ORF2_-138 to_-86, LINE-1_ORF1_-44 to_-1, PSIV_IGR, PV_type1_Mahoney, PV_type3_Leon, REV-A, RhPV_5 NCR, RhPV_IGR, SINV1_IGRpred, SV40_661-830, TMEV, TMV_UI_IRESmp228, TRV_5 NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, ATIR_var1, ATIR_var2, AT1R_var3, ATIR_var4, BAG1_p36delta236nt, BAG1_p36, BCL2, BiP_-222_-3, c-IAP1_285-1399, c-IAP1_1313-1462, c-jun, c-myc, Cat-1224, CCND1, DAPS, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIF1a, hSNM1, Hsp101, hsp70, hsp70, Hsp90, IGF2_leader2, Kv1.4_1.2, L-myc, LamB1_-335-1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, p53_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A_-133_-1, XIAP_5-464, XIAP_305-466, or YAP1. In some embodiments, the IRES element comprises a synthetic IRES, for instance, (GAAA)16 or GAAAGAAAGAAAGAAAGAAAGAAAGAAAGAAA GAAAGAAAGAAAGAAAGAAAGAAAGAAAGAAA (SEQ ID NO: 02, (PPT19)4, KMI1, KMI1, KMI2, KMI2, KMIX, X1, or X2.

In some embodiments, the circular polyribonucleotide includes at least one IRES flanking at least one (e.g., 2, 3, 4, 5 or more) expression sequences. In some embodiments, the IRES flanks both sides of at least one (e.g., 2, 3, 4, 5 or more) expression sequences. In some embodiments, the circular polyribonucleotide includes one or more IRES sequences on one or both sides of each expression sequence, leading to separation of the resulting peptide(s) and or polypeptide(s).

Examples of IRES sequences include but are not limited to:

from crTMV:
(SEQ ID NO: 03)
UUCGUUUGCUUUUUGUAGUAUAAUUAAAUAUUUGUCAUAUAAGAGA
UUGGUUAGAGAUUUGUUCUUUGUUUGAUC;
[75 nt]
from LINE1:
(SEQ ID NO: 04)
CGCAUUAUCUCUCCACGAAUCCAGCCCUUCAAAGGAUAAUAACAGA
AAAAAACG;
[54 nt]
Rbm3:
(SEQ ID NO: 05)
UUUAUAAUUUCUUCUUCCAGAAUC;
(24 nt)
Apaf-1:
(SEQ ID NO: 06)
TAGGCGCAAAGGCTTGGCTCATGGTTGACAGCTCAGAGAGAGAAAGA
TCTGAGGGA
(56 nt)
or
(SEQ ID NO: 12)
UAGGCGCAAAGGCUUGGCUCAUGGUUGACAGCUCAGAGAGAGAAAGA
UCUGAGGGA;
and
c-Myc:
(SEQ ID NO: 07)
GGGGACTTTGCACTGGAACTTACAACACCCGAGCAAGGACGCGACTCT
(48 nt)
or
(SEQ ID NO: 13)
GGGGACUUUGCACUGGAACUUACAACACCCGAGCAAGGACGCGACUCU.

### Protease Cleavage Sites

In some aspects, the mini-circRNAs include nucleic acids sequence that encode one or more protease cleavage sites. If a plurality of protease cleavage amino acid sequences are present, they may be the same or different. This aspect is typically present when the circRNA comprises a region comprising a plurality of antigenic sequences in order to promote or ensure pre-determined post-translational antigen processing. In such aspects, nucleic acids sequences that encode protease cleavage sites are located between each separate, individual antigenic sequence; or between groups of antigenic sequences if it is desired to present some antigenic sequences to the immune system as a single entity, e.g., in the form of a polyvalent chimeric peptide or polypeptide that comprises multiple antigenic sequences, or possibly to present a conformational epitope. Whatever the design, upon translation of the encoded peptide/polypeptide sequences, the peptides/polypeptides are cleaved by endogenous proteases and the encoded peptides/polypeptides are separated into smaller segments. The cleavage sequences may be susceptible to cleavage by any endogenous protease, including but not limited to mammalian aspartic, cysteine, metallo-, serine and threonine proteases. Examples of amino acid sequences of site-specific protease cleavage sites that may be encoded include but are not limited to: AYY, 2A self-cleavage peptides, endoplasmic reticulum-homing signal peptides, etc. In some aspects, the site-specific protease cleavage site is the AYY peptide. Without being bound by theory, the peptides expressed as a rolling concatemer are processed by cells into small fragments for antigen presentation. Thus, protease cleavage sites or other linkers may be used in some embodiments but are not required for others, including multi-antigen vectors.

In these aspects, the plurality of antigenic sequences may each be the same, or each may differ from all other antigenic sequence of the plurality, or some antigenic sequence may be repeated 2 or more times within the coding region. Further, the antigen encoding sequences may be in any order or grouping. For example, for circRNA that encodes six different antigenic sequences A, B, C, D, E and F, the order may be: ABCDEF; BCDEFA; CDEFAB; etc. and all possible permutations thereof. Further, if some (or all) antigenic regions are repeated, the repeats may also be in any order, and some antigenic sequences may be present in more copies than others, for example: BBCCDDEEFFAA; CCCCDDEEFFAABBBB; CDCDEFEFABAB; ABCDEFABCDEF; etc. and all other possible arrangements.

### Stop Codons

In molecular biology (specifically protein biosynthesis), a stop codon (or termination codon) is a codon (nucleotide triplet within messenger RNA) that signals the termination of the translation process of the current protein. Exemplary stop codons that are used in the present circRNAs include but are not limited to: UAG, UAA, and UGA as well as alternatives that have been found in the mitochondrial genomes of vertebrates and in Scenedesmus obliquus and Thraustochytrium such as AGA, AGG, UCA and UUA.

In some aspects, stop codons are not included in the circRNA. In the event of non-stopping RCT, a prerequisite is that the number of nucleotides in a circRNA is a multiple of 3 to ensure the synthesis of the correct peptide sequence without a “frameshift” following the first round of translation.

Other features of circular RNA are described, for example, in issued U.S. Pat. No. 11,160,822, the complete contents of which is hereby incorporated by reference in entirety.

### Pharmaceutical Compositions

The compounds described herein are generally delivered (administered) as a pharmaceutical or therapeutic composition. As used herein, “pharmaceutical composition” refers to a composition suitable for administration to a subject animal, including humans. In the present context, a pharmaceutical composition comprises a pharmacologically effective amount of at least one type of mini-circRNA molecule and a pharmaceutically acceptable carrier, solvent or excipient. Accordingly, pharmaceutical compositions of the present invention encompass any composition made by admixing at least one mini-circRNA in accordance with the present invention and a pharmaceutically acceptable carrier.

A “vaccine” composition (or a “composition for eliciting an immune response) as used herein refers to a composition comprising at least one mini-circRNA molecule as described herein and a pharmaceutically acceptable carrier, solvent, excipient and/or an adjuvant.

Such pharmaceutical and vaccine compositions generally comprise a plurality of at least one type of the disclosed mini-circRNAs, i.e., one or more than one (a plurality) of different mini-circRNAs (e.g., 2 or more such as 2, 3, 4, 5, 6, 7, 8, 9, 10 or more) may be included in a single formulation. Each of the different types encodes a different antigen or group of antigens. Accordingly, the present invention encompasses such formulations/compositions. The compositions generally include one or more substantially purified mini-circRNAs as described herein, and a pharmacologically suitable (physiologically compatible) carrier. In some aspects, such compositions are prepared as liquid solutions or suspensions, or as solid forms such as tablets, pills, powders and the like. Solid forms suitable for solution in, or suspension in, liquids prior to administration are also contemplated (e.g., lyophilized forms of the compounds), as are emulsified preparations. In some aspects, the liquid formulations are aqueous or oil-based suspensions or solutions. In some aspects, the active ingredients are mixed with excipients which are pharmaceutically acceptable and compatible with the active ingredients, e.g., pharmaceutically acceptable salts. Suitable excipients include, for example, water, saline, dextrose, glycerol, ethanol and the like, or combinations thereof. In addition, a composition may contain minor amounts of auxiliary substances such as wetting and/or emulsifying agents, pH buffering agents, preservatives, and the like. In some aspects, it is desired to administer an oral form of the composition so various thickeners, flavorings, diluents, emulsifiers, dispersing aids or binders and the like are added. The compositions of the present invention may contain any such additional ingredients so as to provide the composition in a form suitable for administration. The final amount of compound in the formulations varies but is generally from about 1-99%. Still other suitable formulations for use in the present invention are found, for example in Remington's Pharmaceutical Sciences, 22nd ed. (2012; eds. Allen, Adejarem Desselle and Felton).

A “pharmaceutically acceptable carrier” refers to a clinically useful solvent, dispersion medium, coating, isotonic and absorption delaying agent, buffer, and excipient, such as a phosphate buffered saline solution (PBS), aqueous solutions of dextrose or mannitol, and emulsions, such as an oil-in-water or water-in-oil emulsions, and various types of wetting agents, immunostimulators, and/or adjuvants. Pharmaceutical carriers useful for the composition depend upon the intended mode of administration of the active agent. Some examples of materials which can serve as pharmaceutically acceptable carriers include, but are not limited to, ion exchangers, alumina, aluminum stearate, lecithin, serum proteins (such as human serum albumin), buffer substances (such as Tween® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions. In addition, other non-toxic compatible lubricants such as sodium lauryl sulfate and magnesium stearate, as well as coloring agents, releasing agents, coating agents, sweetening, flavoring and perfuming agents, preservatives and antioxidants may also be present in the composition, according to the judgment of the formulator.

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are generally preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use, examples of which include but are not limited to: ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

Since single-stranded RNA is identified as “foreign” in mammalian cells, the disclosed mini-circRNAs are inflammatory and self-adjuvanting. However, the optional use of additional adjuvants in a pharmaceutical composition is not precluded. An adjuvant is a substance which enhances the body's immune response to an antigen. Many are known in the art and can be utilized in the vaccine compositions of the present invention, including but not limited to: alum (aluminum phosphate, aluminum hydroxide, aluminum potassium sulfate), a cross-linked polyacrylic acid polymer, dimethyldioctadecylammonium bromide (DDA), lactoferrin, an IFN-gamma derivative, a non-ionic detergent, a vegetable oil, surface active substances (including lysolecithin, pluronic polyols, polyanions), various peptides, oil or hydrocarbon emulsions (e.g., oil-in-water emulsion), keyhole limpet hemocyanins, squalene based adjuvants (such as MF59), montanide, RIM adjuvant, complete Freud's adjuvant and incomplete Freud's adjuvant, MPL, muramyl dipeptide, TLR ligand based adjuvants, CpG oligonucleotides, non-CpG oligonucleotides, saponins such as QS-1, ISCOM, ISCOMATRIX, vitamins, and immunomodulants such as cytokines, and the like. Also includes is the saponin adjuvant described in published US patent application 20210228709; rBCG as described in published US patent application 20210222179; Dectin-2 ligand vaccine adjuvant described in published US patent application 20210205445; and vaccine adjuvants described in published US patent application 20210187104. The entire contents of all published US patent applications listed herein are hereby incorporated by reference in entirety.

Formulations suitable for a particular mode of administration are also encompassed, e.g., liquids for injection; pills, capsules, liquids, etc. for oral administration; creams, ointments and suppositories for transmucosal administration; etc. In some aspects, such as for the treatment of cancer, the compounds are incorporated into an implant (e.g., a biodegradable implant) to elicit an immune response to cancer cells systemically or at or near the site of a tumor.

In preferred aspects, lipid nanoparticles (LNPs) are used to deliver circRNA to lymph nodes and antigen-presenting cells (APCs) in vivo. The circRNAs stimulate proinflammatory immune responses and express antigens for adaptive immune modulation. Lipid nanoparticles are spherical vesicles made of ionizable lipids as well as other lipids and cholesterol. Ionizable lipids are positively charged at low pH (enabling RNA complexation) and neutral at physiological pH (reducing potential toxic effects, as compared with positively charged lipids, such as liposomes). Owing to their size and properties, lipid nanoparticles are taken up by cells via endocytosis, and the ionizability of the lipids at low pH likely enables endosomal escape, which allows release of the cargo into the cytoplasm. In addition, lipid nanoparticles usually contain a helper lipid to promote cell binding, cholesterol to fill the gaps between the lipids, and a polyethylene glycol (PEG) to reduce opsonization by serum proteins and reticuloendothelial clearance.

In some embodiments, such lipid nanoparticles comprise a cationic lipid and one or more excipient selected from neutral lipids, charged lipids, steroids and polymer conjugated lipids (e.g., a pegylated lipid). In some embodiments, the mini circ RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g., an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

The LNP may comprise any lipid capable of forming a particle to which the one or more mini circRNAs are attached, or in which the one or more mini circRNAs are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Choi), N-(1-(2,3-dioleoyloxy)propyl)-N-2-(sperminecarboxamido)ethyl)-N,N-dimethy-lammonium trifluoracetate (DOSPA), dioctadecylamidoglycyl carboxy spermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN™ (commercially available cationic liposomes comprising DOTMA and 1,2-dioleoyl-sn-3-phosphoethanolamine (DOPE), from GIBCO/BRL, Grand Island, N.Y.); LIPOFECTAMINE™. (commercially available cationic liposomes comprising N-(1-(2,3-dioleyloxy)propyl)-N-(2-(sperminecarboxamido)ethyl)-N,N-dimethyl-ammonium trifluoroacetate (DOSPA) and (DOPE), from GIBCO/BRL); and TRANSFECTAM™ (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, SM-102, ALC-315, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids. In certain embodiments, the LNP comprises one or more additional lipids which stabilize the formation of particles during their formation. Suitable stabilizing lipids include neutral lipids and anionic lipids.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In some embodiments, the LNPs comprise a neutral lipid selected from DSPC, DPPC, DMPC, DOPC, POPC, DOPE and SM.

In various embodiments, the molar ratio of the cationic lipid to the neutral lipid ranges from about 2:1 to about 8:1.

In various embodiments, the LNPs further comprise a steroid or steroid analogue. In certain embodiments, the steroid or steroid analogue is cholesterol. In some of these embodiments, the molar ratio of the cationic lipid to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GMi). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols.

Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(co-methoxy(polyethoxy)ethyl)bu-tanedioate (PEG-S-DMG), a pegylated ceramide (PEG-cer), or a PEG dialkoxypropylcarbamate such as Q-methoxy(polyethoxy)ethyl-N-(2,3-di(tetradecanoxy)propyl)carbamate or 2,3-di(tetradecanoxy)propyl-N-(co-methoxy(polyethoxy)ethyl)carbamate. In various embodiments, the molar ratio of the cationic lipid to the pegylated lipid ranges from about 100:1 to about 25:1.

In certain embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In certain embodiments, the LNP comprises one or more targeting moieties which are capable of targeting the LNP to a cell or cell population. For example, in one embodiment, the targeting moiety is a ligand which directs the LNP to a receptor found on a cell surface.

In certain embodiments, the LNP comprises one or more internalization domains. For example, in one embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in WO2016176330A1, U.S. Patent Application Publication No. US20120276209, Semple et al., 2010, Nat Biotechnol., 28(2): 172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tarn et al., 2013, Nanomedicine, 9(5): 665-74, each of which are incorporated by reference in their entirety. Additional examples of LNPs are described in issued U.S. Pat. Nos. 11,168,051; 11,026,894; 10,940,207; 10,342,760; and 10,307,490, the complete contents of each of which is hereby incorporated by reference in entirety.

Those of skill in the art will recognize that the LNPs may be delivered as per any of the formulations and methods described herein, e.g., enclosed in a capsule that dissolves upon oral administration, prepared for injection e.g., in a suspension, etc. Further, such formulations may include one or more cryoprotectants. In some embodiments, the cryoprotectant is added to the LNP solution prior to the lyophilization and storage. In some embodiments, the cryoprotectant comprises one or more cryoprotective agents, and each of the one or more cryoprotective agents is independently a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof), or any combination thereof. In some embodiments, the cryoprotectant comprises sucrose.

### Uses

The vaccine compositions described herein are used prophylactically and/or therapeutically. A “prophylactic” treatment is a treatment administered to a subject who does not exhibit signs of an infection or disease for the purpose of reducing the likelihood of the infection or disease, decreasing the risk of developing pathology from the infection or disease, decreasing the severity of the infection or disease, decreasing the duration of an infection or disease should one occur, etc. A “therapeutic” treatment is a treatment administered to a subject who already exhibits one or more signs or symptoms of infection or disease. However, in some cases, e.g., for treating cancer such as breast or colon cancer, the disease may be silent (symptomless) and may be discovered and diagnosed by a screening technique such as a mammogram, colonoscopy, etc., and/or by biopsy. Administration of the mini-circRNAs is generally for the purpose of reducing the severity of infection or disease and/or shortening the duration of infection or disease and/or reducing or eliminating one or more signs or symptoms of an infection or disease and/or extending the life span of a recipient and/or increasing the quality of life of a recipient, etc. Those of skill in the art will recognize that the outcome of a treatment may be to completely cure or eradicate the disease. However, much benefit can accrue even if a “cure” is not achieved, e.g., by reducing or ameliorating at least one symptom of a disease, extending the life span or increasing the quality of life of a patient, etc. In one aspect, a therapeutic vaccine for cancer may be administered before surgery to reduce the burden of a tumor (neoadjuvant). In another aspect, a therapeutic vaccine for cancer may be administered after surgery to reduce the residual disease and/or reduce the chances of recurrence.

### Administration

The therapeutic formulations disclosed herein are administered in vivo by any suitable route including but not limited to: inoculation or injection (e.g., intravenous, intraperitoneal, intramuscular, subcutaneous, intradermal, intra-aural, intra-articular, intra-mammary, and the like); orally; topical application (e.g., transdermally); by absorption through epithelial or mucocutaneous linings (e.g., intranasally, orally, vaginally, rectally, via gastrointestinal mucosa, and the like); intraorbitally; by implantation; by inhalation (e.g., as a mist or spray); intrathecally, intraventricularly, etc. Typical modes of administration include but are not limited to: parenteral administration, including subcutaneous, intramuscular, intravenous, intraperitoneal, or intratumoral injection; administration to one or more lymph nodes; transdermal and transmucosal administration; inhalation or nasal spray; etc. In preferred aspects, administration is by inoculation or injection or inhalation or nasal spray. In some embodiments, administration is via intravenous infusion. In some aspects, administration is directly into one or more lymph nodes, e.g., by injection.

The amount of the mini-circRNA that is administered to a subject is generally a therapeutically effective dose, e.g., a does sufficient to prevent or ameliorate at least one symptom of a disease or disorder. An “effective amount” is at least the minimum amount required to effect a measurable improvement or prevention of a particular disorder. An effective amount herein may vary according to factors such as the disease state, age, sex, and weight of the patient, and the ability of the mini-circRNA to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The exact amount in a “dose” of mini-circRNA can vary, ranging from about 0.01 to about 10,000 μg for each subject, such as from about 0.01, 0.1, 1.0, 10, 20, 34, 40, 50, 60, 70, 80, 90 or 100 μg, or from about 100, 200, 300, 400, 500, 600, 700, 800, 900 or 1,000 μg, or from about 1000, 2,000, 3,000, 4,000, 5,000, 6,000, 7000, 8,000, 9,000 or 10,000 for each subject. The amounts are generally determined by a skilled practitioner, e.g., a physician, in view of clinical trial results and/or FDA recommendations.

Similarly, the frequency or protocol of administration may vary. For example, as a prophylactic vaccine, the mini-circRNA may be administered e.g., once or multiple times by administering booster shots that are given, e.g., 1-8 weeks after the first dose and/or 1-8 months after the first dose, and thereafter at 6-month or yearly intervals. In a year, a total of e.g., 2-3 or more doses may be administered, e.g., up to about 3, 4, 5, or 6 or more if monthly dosing is advised.

For the treatment of cancer, the dosing may be the same or may be more frequent, e.g., daily for a period of time, and/or weekly for a period of time and/or monthly and/or every 6-10 weeks, etc. Further, the dosing may be integrated into the treatment plan of the individual, e.g., to alternate with or coincide with chemotherapy, radiation therapy, surgery, check point inhibitor therapy, antibody therapy, etc. Exemplary cancer treatment protocols are described, for example, in published US patent application 20210346485, the complete contents of which is hereby incorporated by reference in entirety.

A “subject” or a “patient” as used herein refers to any animal classified as a mammal, including humans, domestic and farm animals, and zoo, sports, or pet animals, such as dogs, horses, cats, cows, etc. However, veterinary uses are also encompassed. A subject to whom the vaccine is administered may be a mammal, such as a human. However, veterinary applications of this technology are also encompassed so that companion pets (e.g., dogs, cats, ferrets, etc.); and/or working animals (cows, horses, camels, donkeys, etc.); and/or animals in preserves, laboratories or zoos, etc.; and even non-mammals such as poultry, pet birds, amphibians, reptiles, fish, etc.; can be immunized or treated using the mini-circRNAs.

The compositions may be administered in conjunction with other treatment modalities such as but not limited to substances that boost the immune system, various chemotherapeutic agents, antibiotic agents, pain medication, and the like.

In further embodiments, e.g., when the disorder is cancer, the additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation.

If the mini circRNA encodes a multivalent sequence (i.e., encodes a plurality of immunogens or a plurality of repeats of an immunogen) then administration of the mini-circRNA disclosed herein results in or causes translation of the encoded immunogenic sequences as concatemeric peptides via rolling circle translation (RCT). Individual peptides may be separated by protease cleavage of the protease cleavage sites present between each immunogen.

The amino acid sequences that are encoded by the mini-circRNAs are translated in vivo for extended periods of time. For example, in some aspects they are translated for at least 1-7 days (e.g., for at least 1, 2, 3, 4, 5, 6, days or a week). In other aspects, they are translated for 1-4 weeks, such as for about 1, 2, 3 or 4 weeks (e.g., a month). Such long periods of translation advantageously provide ample opportunity for the immune system of a vaccine recipient to become activated and produce antibodies and other immune factors.

Diseases and Disorders that are Treated

### Proliferative Disorders

In some aspects, the disease or disorder that is prevented or treated is a “cell proliferative disorder” (proliferative disorder) and the antigens that are encoded are cancer antigens/epitopes. According to the invention, the terms “tumor antigen”, “tumor-expressed antigen”, “cancer antigen” and “cancer-expressed antigen” are equivalents and are used interchangeably herein. “Cell proliferative disorders” refers to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein. Examples of cancers that are prevented or treated include but are not limited to: melanoma, non-small cell lung cancer, bladder cancer, colorectal cancer, triple negative breast cancer, renal cancer, and head and neck cancer. In some embodiments, the cancer is locally advanced or metastatic melanoma, non-small cell lung cancer, bladder cancer, colorectal cancer, triple negative breast cancer, renal cancer, or head and neck cancer. In some embodiments, the cancer is selected from the group consisting of non-small cell lung cancer, bladder cancer, colorectal cancer, triple negative breast cancer, renal cancer, and head and neck cancer. In some embodiments, the cancer is locally advanced or metastatic non-small cell lung cancer, bladder cancer, colorectal cancer, triple negative breast cancer, renal cancer, or head and neck cancer.

In some embodiments, the cancer is melanoma. In some embodiments, the melanoma is cutaneous or mucosal melanoma. In some embodiments, the melanoma is cutaneous, mucosal, or acral melanoma. In some embodiments, the melanoma is not ocular or acral melanoma. In some embodiments, the melanoma is metastatic or unresectable locally advanced melanoma. In some embodiments, the melanoma is stage IV melanoma. In some embodiments, the melanoma is stage IIIC or stage IIID melanoma. In some embodiments, the melanoma is unresectable or metastatic melanoma. In some embodiments, the method provides adjuvant treatment of melanoma. In some embodiments, the cancer (e.g., melanoma) is previously untreated. In some embodiments, the cancer is previously untreated advanced melanoma. In some embodiments, the melanoma antigen is gp75 and/or high molecular weight melanoma antigen and/or melanoma-associated antigen p97.

In some aspects, the disease that is prevented or treated is breast cancer, such as triple negative breast cancer, and the particular antigenic regions or epitopes include, for example: a set of tumor antigens comprising one or more of CXorf61, CAGE1 and PRAME.

In some aspects, the disease that is prevented or treated is prostate cancer and the antigens include prostatic acid phosphate, prostate specific antigen and/or prostate specific membrane antigen; and/or the particular antigenic regions or epitopes include, for example those that are described in published US patent application 20200222478, the entire contents of which is herein incorporated by reference in entirety.

In some aspects, the disease that is prevented or treated is the recurrence and/or metastasis of a cancer caused by the escape of cancer stem cells, and the antigens include one or more of amino acid sequence coded by Or7c1, Dnajb8, Sox2, Smcp, Ints1, Kox12, Mdf1, FLJ13464, 667J232, Surf6, Pcdh19, Dchs2, Pcdh21, Gal3st1, Rasl11b, Hes6, Znf415, Nkx2-5, Pamci, Pnmt or Scgb3a1, or a portion thereof, as described in published US patent application 20110262358, the entire contents of which is herein incorporated by reference in entirety.

In some aspects, the disease that is prevented or treated is ovarian cancer and the antigen is ovarian carcinoma antigen (CA125).

In some aspects, the antigen is the KS ¼ pan-carcinoma antigen.

### Infectious Diseases

In other aspects, the disease or disorder that is prevented or treated is an infectious disease. The infectious disease may be caused by any of a variety of microbes and/or parasites that have antigens, epitopes, antigenic regions, etc. that can be encoded by the mini circ RNAs disclosed herein. Examples of such microbes are well-known in the art.

In some aspects, the infectious agent is a coronavirus. The coronavirus may be any of the four genera: Alphacoronaviruses and betacoronaviruses (which infect mammals) or gammacoronaviruses and deltacoronaviruses which primarily infect birds. The genus Alphacoronavirus includes species: Alphacoronavirus 1 (TGEV, Feline coronavirus, Canine coronavirus), Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2 and Scotophilus bat coronavirus 512. The genus Betacoronavirus include the species: Betacoronavirus (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-COV, SARS-CoV-2 and variants thereof) and Tylonycteris bat coronavirus HKU4. The genus Gammacoronavirus includes the species: Avian coronavirus and Beluga whale coronavirus SW1. The genus Deltacoronavirus includes the species: Bulbul coronavirus HKU11 and Porcine coronavirus HKU15.

In some aspects, the infectious agent is SARS-CoV-2, which mainly attacks the lower respiratory system. SARS-CoV-2 can also infect the gastrointestinal system, heart, kidney, liver, and central nervous system, leading to multiple organ failure.

### Articles of Manufacture or Kits

Further provided herein is an article of manufacture or a kit comprising at least one type of mini-circRNA and/or pharmaceutical preparation as described herein. In some embodiments, the article of manufacture or kit further comprises package insert comprising instructions for using the mini-circRNA and/or pharmaceutical preparation to prevent or treat the occurrence or progression of a disease or disorder as described herein, e.g., to enhance immune function of an individual susceptible to the disease or disorder.

The mini-circRNA and/or pharmaceutical preparation is generally provided in a container. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including buffers, diluents, filters, needles, syringes, and package inserts with instructions for use. In some embodiments, the article of manufacture further includes one or more of another agent e.g., a chemotherapeutic agent, an anti-neoplastic agent, etc.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1

Design, synthesize, and characterize mini circRNA for a model antigen SIINFEKL (SEQ ID NO:08).

Design of circRNA

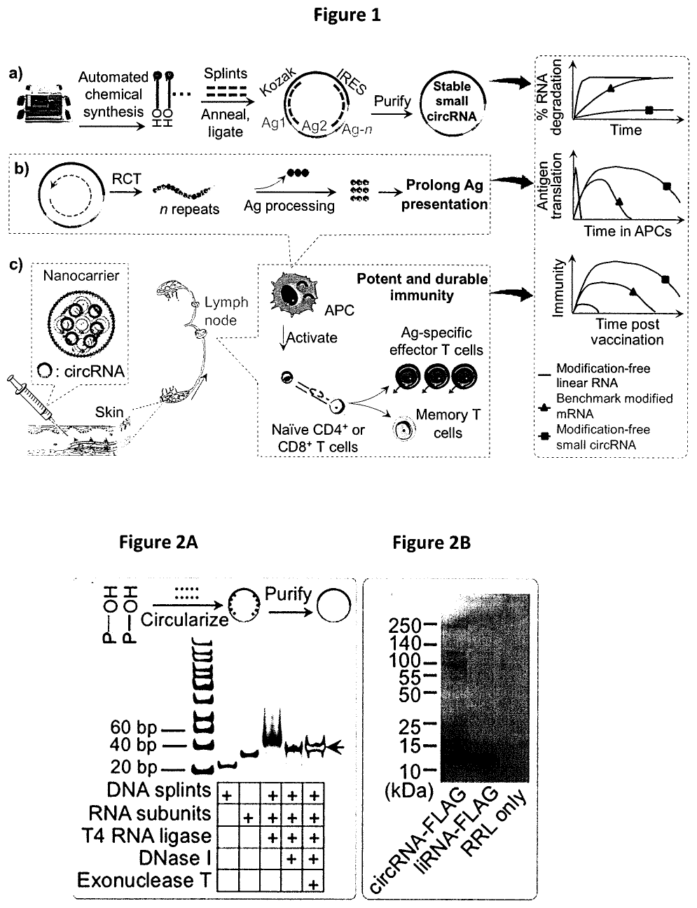

CircRNA consists of three parts: 1) internal ribosome entry site (IRES) that initiates protein translation in a cap-independent manner, 2) Kozak sequence (GCCACCAUG) which initiates protein translation in eukaryotic systems; and 3) a SIINFEKL-coding region (GAG AGUAUAAUCAACUUUGAAAAACUG, SEQ ID NO:09). The ovalbumin (OVA)-derived peptide antigen, SIINFEKL (SEQ ID NO:08), was used as a model because it elicits potent CD8⁺ T cell responses and can be easily replaced with other peptide antigens. To select an efficient IRES, we designed three circRNAs using the following three IRES for murine (5′-3′) translation: LINE1: CGC AUU AUC UCU CCA CGA AUC CAG CCC UUC AAA GGA UAA UAA CAG AAA AAA ACG (SEQ ID NO:04); Rbm3: UUU AUA AUU UCU UCU UCC AGA AUC (SEQ ID NO:05) and crTMV: UUC GUU UGC UUU UUG UAG UAU AAU UAA AUA UUU GUC AUA UAA GAG AUU GGU UAG AGA UUU GUU CUU UGU UUG AUC (SEQ ID NO:03). These linear RNAs were synthesized commercially. To leverage automated RNA synthesis and minimize unwanted immune responses by non-essential RNA, we designed minimal circRNA with short IRES, Kozak sequence (GCCACCAUGG, SEQ ID NO:10), and peptide antigen-encoding RNA (FIGS. 1A-C).

### Circularization of Linear RNA Using T4 RNA Ligase 1

Using MHC-I-restricted ovalbumin (OVA) (SIINFEKL, SEQ ID NO: 08) that elicits CD8⁺ T cell response, we synthesized circRNA by circularizing 5′-phosphate-RNA using T4 RNA ligase I and DNA splints, which facilitate ligation of RNA, prior to linear/lariat RNA removal by RNA exonuclease T, DNA removal by DNase I, HPLC purification, and verification by gel electrophoresis (FIG. 2A). To study peptide translation by circRNA, we designed a circRNA encoding a FLAG tag (DYKDDDDK, SEQ ID NO:11). As shown by in vitro translation in cell-free rabbit reticulocyte lysate, circRNA produced FLAG concatemers likely via rolling circle translation, as indicated by a series of products >10-fold larger than FLAG in western blot (FIG. 2B).

Specifically, circRNAs were synthesized by circularizing the 5′-end phosphate-modified linear RNAs using T4 RNA ligase I (New England Biolabs) using complementary DNA splints as ligation templates (FIG. 2A). The DNA splints are short linear DNA deoxyoligonucleotides that are partially complementary to the terminals of linear RNAs. One DNA splint hybridize with at least two RNA terminals so as to bring these RNA terminals into close proximity, which allow the following conjugation of these two RNA terminals in a process of ligation. In practice, all linear RNAs and DNA splints were mixed together, followed by annealing to allow the hybridization of DNA splints of parts of linear RNA, which generate a circular RNA that are not yet covalent linked at the RNA nicking sites, which the DNA splints bring into close proximity. After annealing with DNA splints (3-5 μM), 1 μM linear RNA was incubated with 1 μl (10 units) T4 RNA ligase 1 in a mixture of 50 mM Tris-HCl (pH 7.5), 10 mM MgCl₂, 1 mM DTT, 0.5 μl RNase inhibitor (40μ/ul), 10% PEG8000, and 50 μM ATP at 25° C. for 2 h.

### Circularity Check of the RNA Using DNase I and RNA Exonuclease T

RNA (1 μM) was incubated with DNase I (2 unit/μL; New England Biolabs) in DNase I Reaction Buffer at 37° C. for 10 min. The reaction mixture (5 μL) was analyzed by 6% denaturing PAGE. Then the solution was incubated with 3′ to 5′ RNA Exonuclease T (5 unit/μL; New England Biolabs) at 25° C. for 30 min, and then incubated at 65° C. for 20 minutes. circRNAs were verified by agarose gel electrophoresis (FIG. 2B). This assay also removes the DNA scaffold and any RNA that is not covalently circularized.

Liposomal circRNA Synthesis and Characterization

The lipid components including DOTAP/DSPE-PEG/DOPE/Chol at a molar ratio of 1:0.05:1:0.5, were dissolved in the chloroform. After the evaporation of the solvent under vacuum using a rotary evaporator, a thin lipid film formed at the bottom of the flask and was subsequently hydrated with RNase-free water containing circRNA (with the charge molar ratios of cationic lipids and nuclei acids at 10) by sonicating for 30 min and vigorous stirring every 10 min until the suspension was homogenized. The hydrated liposome suspension was extruded 11 times through a 200 nm polycarbonate membrane in a mini-extruder (Avanti Polar Lipids). The liposome preparation was then immersed into liquid nitrogen before lyophilization overnight. The lyophilized powder of liposomes/circRNA complexes were reconstituted in DI water before characterization of their nanoparticle properties using transmission electronic microscope (TEM) for the morphology and dynamic light scattering (DLS) for the size distribution and surface zeta potential.

Excellent Pharmaceutical Stability and Biostability of circRNA

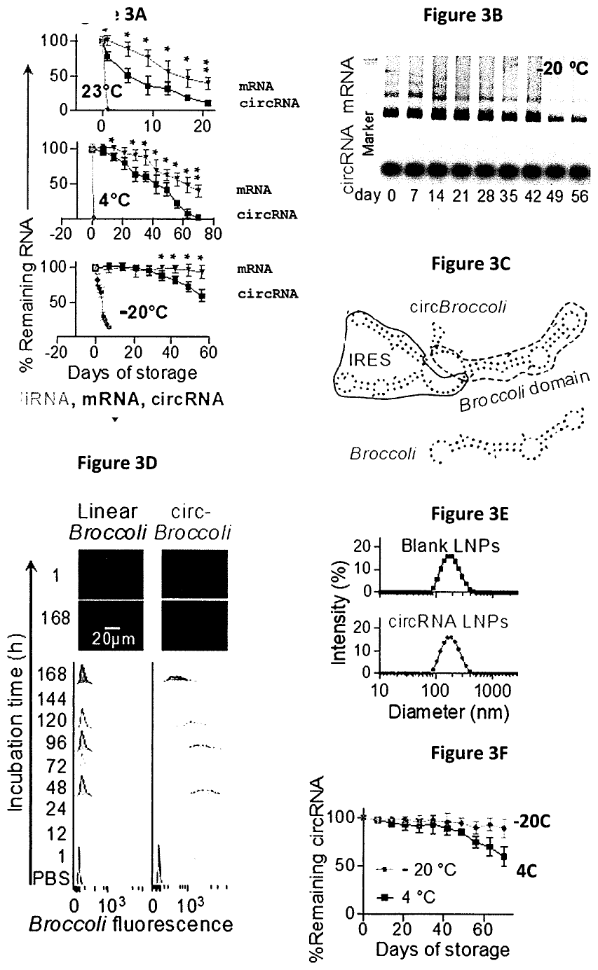

mRNA is easily degraded by ubiquitous RNase in cells, biofluids, and the environment, which limits its shelf-life and in vivo half-life of antigen translation. Current mRNA vaccines have limited stability, despite extensive modifications. Due to the lack of termini, modification-free circRNA resisted exonucleases, the primary RNase for RNA degradation. Remarkably, in storage solutions at −20° C., 4° C., or 23° C. for up to 70 days, circRNA showed superior stability to small liRNA and current state-of-the-art mRNA-OVA that is stabilized by 5′/3′ untranslated regions, A120, and CleanCap® (TriLink) (FIG. 3A-B). To study circRNA stability in live cells, we replaced the antigen-encoding RNA in circRNA with a fluorogenic RNA aptamer, Broccoli, which fluoresces upon binding to DFHBI-1T (FIG. 3C). The linearization of circular Broccoli (circBroccoli) would rapidly degrade Broccoli. After transfection into a line of dendritic cells (DCs) known as DC2.4 cells (7 days), circBroccoli sustained fluorescence despite signal dilution due to cell division, whereas linear Broccoli fluorescence faded away within hours (FIG. 3D). We then studied the pharmaceutical stability of circBroccoli loaded (N/P ratio: 3) in LNPs (50:38.5:10:1.5 molar ratio of SM-102, cholesterol, DSPC, PEG2k-DMG) (FIG. 3E). 70 days after storage in solution at 4 or −20° C. (cryoprotectant sucrose-supplemented), LNPs were transfected to DC2.4 cells (24 h). circBroccoli retained strong fluorescence intensity in DCs, indicating excellent pharmaceutical stability of circRNA-LNPs (FIG. 3F). The great stability of circRNA alleviates the stringent storage or transportation conditions and extends shelf-life relative to current mRNA vaccines.

In Vivo Delivery and Screening of circRNAs for Antigen Expression and Immune Modulation in Mice: Mini circRNA LNPs for Lymph Node Delivery and Intracellular Delivery in DCs and Macrophages

An mRNA vaccine needs to be delivered to lymphoid tissues (e.g., lymph nodes) and APCs (e.g., DCs), yet such delivery is challenged by the poor pharmacokinetics, limited cell uptake, and poor endosome escape of RNA. Nucleic acid therapeutics face multiple delivery barriers at the tissue, cell, as well as endolysosome levels. Nonviral nanocarriers, including LNPs, have been developed to deliver nucleic acids across these barriers. Multiple LNP nucleic acid therapeutics, including siRNA LNPs, have been approved by FDA. Vaccines must be delivered into secondary lymphoid organs (e.g., lymph nodes), where antigens can interact with immune cells and modulate immune responses. LNPs are used to deliver circRNA to lymph nodes and APCs in vivo. LNPs are synthesized with D-Lin-MC3-DMA: DSPC: Cholesterol: PEG-lipid at a molar ratio of 50:10:38.5:1.5, and N:P ratio (positively charged amine (N) to negatively charged phosphate (P)) of 1:1, 5:1, 10:1, and 20:1, respectively. The LNP diameters are controlled to be around 100 nm, and circRNA loading capacity and loading efficiency is determined by agarose gel electrophoresis. The charge ratio with the highest loading efficiency and lowest toxicity in DCs and macrophages is used for further experiments.

### Cell Uptake.

For fluorescence monitoring of circRNA, circRNA was labeled with a fluorophore Cy5 via hybridizing with a partially complementary Cy5-labeled DNA (IDT). Murine DCs were incubated with liposomal circRNA-Cy5, with the corresponding free circRNA-Cy5 as the control for a series of time points. Fluorescence intensities of the resulting cells were then measured by flow cytometry. The intracellular fluorescence intensity and distribution were observed by confocal microscopy in the treated live cells that were stained with Hoechst33342 and LysoTracker-Green. The ability of circRNA to be taken up by cells and then escape the endosome to reach the cytosol is critical for the circRNA coding region to be translated in the cytosol. Cy5 fluorescence intensity ratio was measured (inside/outside endolysosome, I/O) in 50 randomly selected cells.

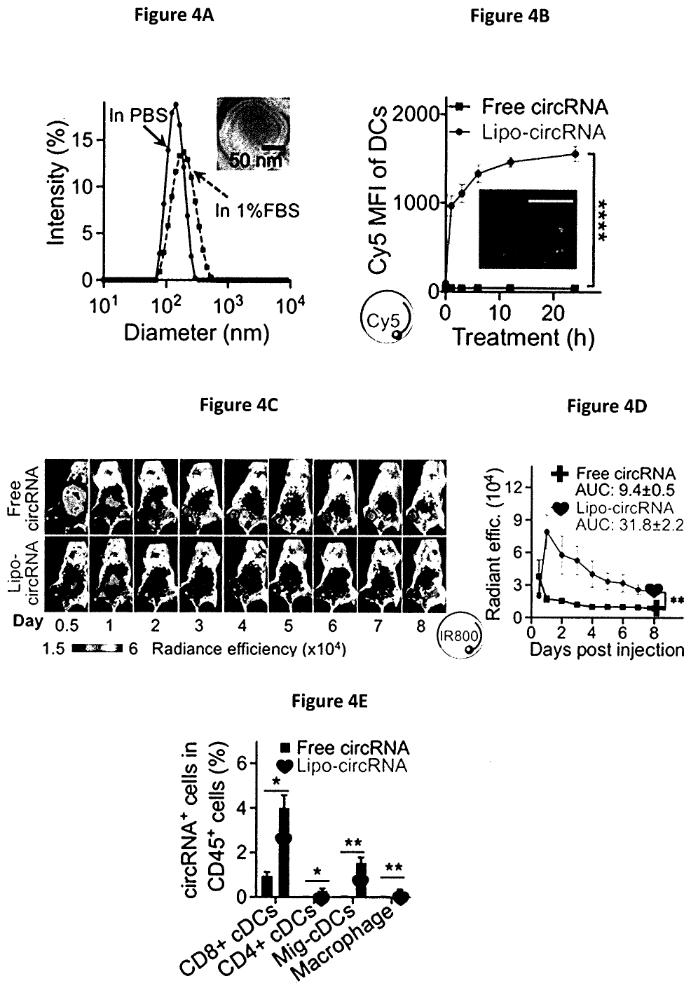

We showed that LNPs and liposome [diameter: ˜120 nm] (FIG. 4A) efficiently delivered circRNA to lymph nodes and APCs. For instance, liposomal circRNA (lipo-circRNA) had ˜95% circRNA encapsulation efficiency [N/P ratio: 3]. After incubation of DC2.4 cells with liposomal vs. free circRNA labeled with a Cy5-cDNA, liposome enhanced circRNA cell uptake and its escape from endosome to cytosol where circRNA can produce peptides (FIG. 4B). Remarkably, subcutaneously (s.c.) injected lipo-circRNA-IR800 (IR800: near-infrared dye) was retained in the draining lymph nodes for at least 8 days in Balb/c mice (FIGS. 4C-D), due to the efficient liposome delivery and the high circRNA biostability. 24 h post injection, liposome promoted circRNA retention in intranodal APCs, especially CD8+ conventional DCs (CDC) pivotal for antigen cross-presentation (FIG. 4E). The efficient circRNA delivery to lymph nodes, APCs, and cytosol is key to its antigen expression and immunomodulation.

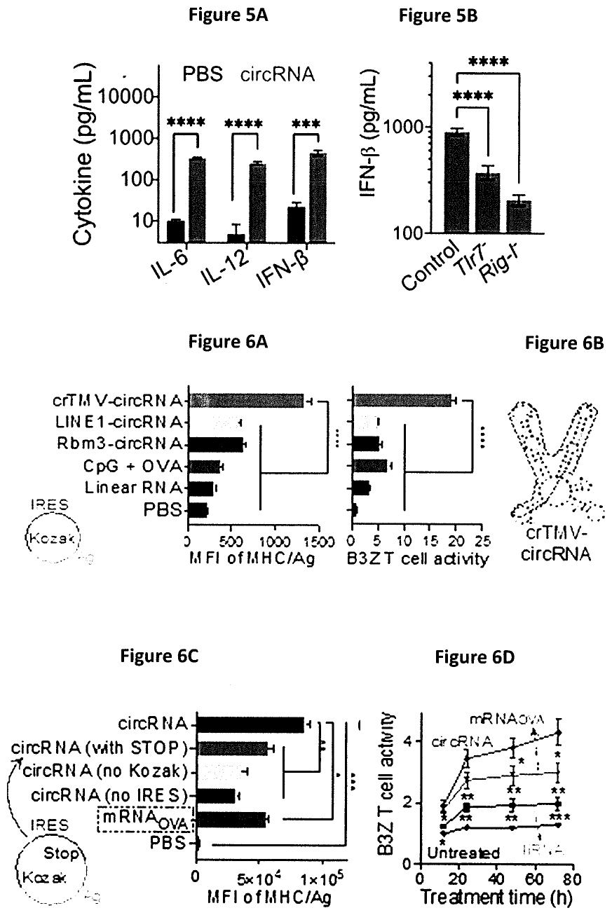

RNA is intrinsically immunostimulatory because ssRNA, dsRNA, and nucleotides activate pattern recognition receptors (PRRs), such as toll-like receptors (TLRs). The resulting innate immune responses provide cytokines and co-stimulation, or signal 2 that is critical to activate naïve T cells. In DC2.4 cells, circRNA-SIINFEKL nanovaccines significantly induced the secretion of proinflammatory factors (IL-6, IL-12) and type I interferon (e.g., IFN-β) (FIG. 5A), suggesting that circRNA vaccines are embedded with a “danger” signal and are self-adjuvanted. As a step to identify immune sensors for circRNA, siRNA silencing of Tlr7 or Rig-I in circRNA-treated DC2.4 cells appeared to significantly reduce IFN-β levels, implicating their potential involvement in sensing small circRNA (FIG. 5B).

### Antigen-Presenting Analysis

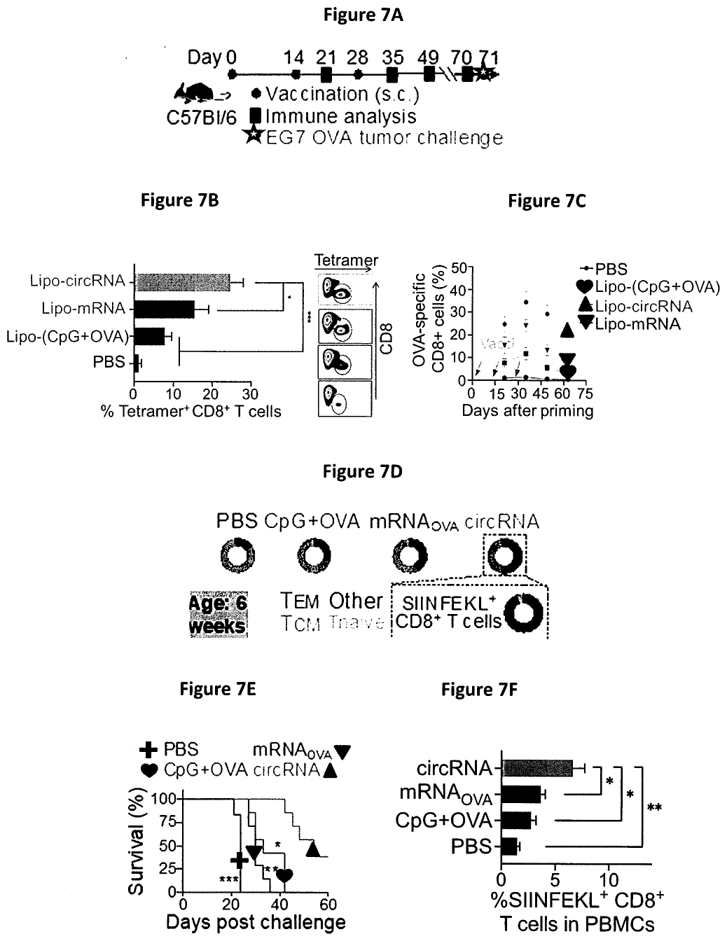

To select an IRES for optimal antigen translation by circRNA-SIINFEKL, we tested three short IRES: 73-mer crTMV, 53-mer LINE1, and 22-mer Rbm3. DC2.4 cells were Lipo3k-transfected with circRNA, linear RNA (liRNA), or CpG+OVA. 24 h later, MHC-I/SIINFEKL-staining of DCs showed that crTMV drove the most efficient antigen presentation (FIGS. 6A-B).

### B3Z Cell Activation Assay

A B3Z cell is a CD8⁺ T-cell hybridoma. Upon recognition of the H-2K^{b}/SIINFEKL complex, B3Z cells will be activated to produce B-galactosidase, which can hydrolyze the substrate into red products. The level of activation of the CD8+ T cells is thus reflected by the color of the solution. To perform this assay, DC2.4 cells are cultured with medium containing liposomal circRNA for a series of time points. The cells are subsequently co-cultured with B3Z cells for 24 h. Then the cells are lysed for 4 h at 37° C. with lysis buffer (PBS with 100 mM 2-mercaptoethanol, 9 mM MgCl₂, 0.2% Triton X-100 and 0.15 mM chlorophenol red-β-D-galactopyranoside). The reaction is stopped by adding the stop buffer (1 M sodium carbonate). CD8+ T cell activation is quantified by measuring the absorbance at 570 nm with 635 nm as a reference wavelength. B3Z cell activation is shown as the normalized optical density (OD) relative to the control group. Consistently, crTMV-circRNA-treated DCs primed SIINFEKL-specific B3Z CD8⁺ T cell hybridoma the most efficiently (FIG. 6A). Further, inserting a stop codon at the 3′-end of antigen-encoder, or knockouts of IRES or Kozak inhibited antigen presentation by circRNA (FIG. 6C). Thus, crTMV was selected for further studies. Remarkably, circRNA outperformed the current state-of-the-art mRNA-OVA modified with 5-methoxyuridine, A120, 5′-/3′-untranslated regions, and CleanCap® (TriLink) for antigen presentation and T cell priming (FIGS. 6C-D).

T Cell Responses to circRNA Vaccines in Mice: Low-Dose circRNA Nanovaccines Induced Potent and Durable T Cell Immunity in Young Adult and Aged Mice

T cell response is pivotal for tumor immunotherapy. First, we studied T cell responses by low-dose model circRNA-SIINFEKL nanovaccines in C57BL/6 mice (5 μg in liposome, days 0, 14, subcutaneous at tail base) (FIG. 7A). This dose is 6-10-fold lower than typical doses of T cell mRNA vaccines. Mouse peripheral CD8⁺ T cells were stained with antigen-specific tetramers to measure the fraction of antigen-specific CD8⁺ T cells. PE-conjugated H-2Kb-SIINFEKL tetramers (manufactured at the NIH Tetramer Core Facility) were used for tetramer staining of OVA-vaccinated mice. Briefly, mice were treated with liposomal circRNA on day 0 and day 14. Blood was collected from the treated mice on day 21. Blood cells were enriched by centrifugation. Red blood cells were lysed using ACK lysis buffer for 10 min at room temperature. Blood clots were removed using a filter. Cells were washed twice in PBS and cells were stained using Zombie Aqua™ Fixable Viability Kit (Biolegent) for 10 min at room temperature. Staining was quenched and cells were washed with FCS buffer (PBS buffer with 0.1% FBS). Cells were then blocked with anti-CD16/CD32 for 10 min, followed by adding a dye-labeled antibody cocktail (anti-CD62L-FITC, anti-CD44-Alexa 647, anti-CD8-APC-Cy7, Tetramer-PE, anti-PD-1-BV421) and stained at room temperature for 30 min. anti-CD62L-FITC and anti-CD44-Alexa 647 were used to stain T cell memory phenotypes. anti-PD-1-BV421 was used to stain immune checkpoint PD-1. Cells were then washed, and 100 μL Cytofix™ was added to each well to resuspend cells, and cells were fixed at 4° C. for 20 min. Cells were then washed with Perm/Wash buffer and resuspended for flow cytometric analysis.

More than two months post priming immunization, mice were challenged with EG7.OVA cells, followed by monitoring the tumor growth and mouse survivals. Tetramer staining showed that circRNA nanovaccines profoundly augmented antigen-specific CD8+ T cells (24%, day 21), in contrast to 15% by the current state-of-the-art 5-methoxyuridine CleanCap® mRNAOVA nanovaccines (TriLink) and 7% by CpG+OVA nanovaccines. A 2^{nd} booster (day 28) further expanded SIINFEKL⁺ CD8⁺ T cells to 35% (day 35), and this frequency remained >20% for 70 days (FIG. 7B, C). circRNA nanovaccines induced CD8+CD62L^{low}CD44⁺ effector memory (TEM) and CD62L^{high}CD44⁺ central memory T cells (TCM), especially SIINFEKL⁺ memory T cells that are key to durable antitumor immunity that can help prevent recurrence (FIG. 7D), allowing circRNA nanovaccines to outperform nanovaccines of mRNAOVA or CpG+OVA upon challenge with EG7.OVA tumor (day 71) (FIG. 7E). In summary these results showed that circRNA vaccine efficiently induced antigen-specific T cells that outperformed modified OVA-encoding mRNA and CpG-adjuvanted protein OVA vaccines, over more than two months after priming immunization; circRNA vaccine induced CD8+ T cell memory, including antigen-specific CD8+ T cell memory; and as a result, circRNA vaccine-treated mice efficiently retarded the growth of EG7.OVA cells and prolonged the survival of the immunized mice.

Due to immunosenescence during aging, the elderly are vulnerable to many types of cancers with limited ability to elicit antitumor immunity.¹¹¹ Vaccines are desired to promote antitumor immunity in the elderly. In aged C57BL/6 mice (1 year), circRNA-SIINFEKL nanovaccines (5 μg; days 0, 14) induced 6.66% SIINFEKL⁺ CD8⁺ T cells in PBMCs (day 21), in contrast to 3.62% by mRNAOVA nanovaccines and 2.75% by CpG+OVA nanovaccines (FIG. 7F). Intracellular IFN-γ/TNF-α staining showed potent functionality of CD8⁺ T cells from vaccinated mice (day 35) (FIG. 7G). circRNA nanovaccines also elicited great CD8+ T cell memory and resisted EG7.OVA cell challenge (FIG. 7H).

### Screening Nanocarriers for Efficient T Cell Responses

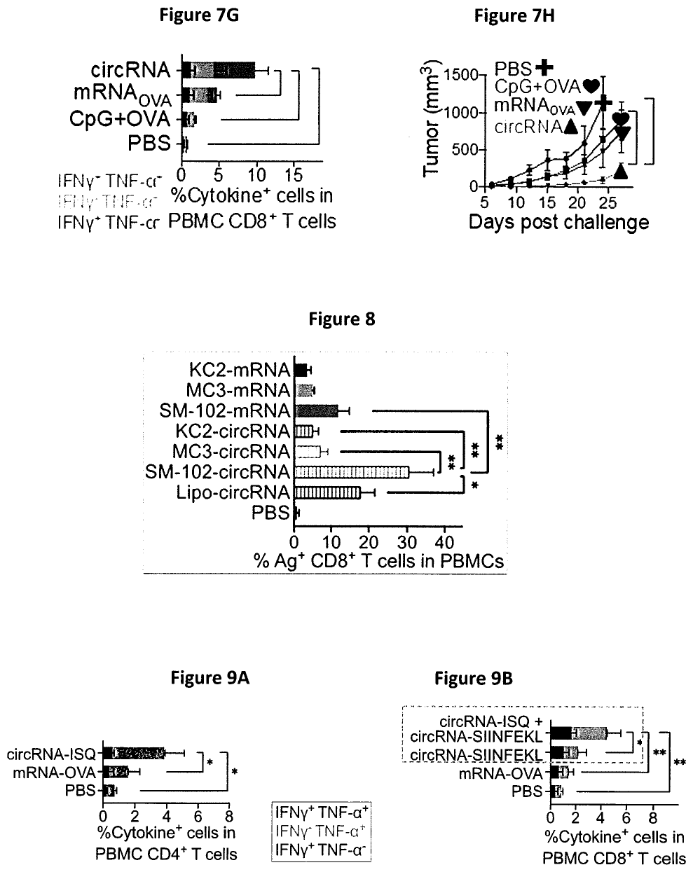

The immunomodulatory efficacy of mRNA vaccines is hinged on their in vivo delivery efficiency. Various ionizable LNPs have been used for long mRNA delivery or systemic siRNA delivery. Yet, small circRNAs are physicochemically distinct from these RNA. For circRNA delivery, we screened liposome as well as LNPs based on ionizable lipids SM-102, Dlin-MC3-DMA, and Dlin-KC2-DMA that are widely used to deliver RNA therapeutics¹ and vaccines. At N/P ratio of 6, we loaded circRNA-SIINFEKL and mRNAOVA into these nanocarriers (LNP synthesis by microfluidic rapid mixing) (d: 80-120 nm). By estimation, 185 circRNA-SIINFEKL copies (113 nucleotides) vs. 14 mRNA copies (1437 nucleotides) were loaded per LNP. We tested these nanovaccines (5 μg RNA; subcutaneous; day 0, 14) to induce T cell response by tetramer staining (day 21). SM-102 LNP showed the highest SIINFEKL⁺ CD8⁺ T cell response for both circRNA and modified mRNA in C57BL/6 mice (FIG. 8). Thus, SM-102 LNP was selected for further study.

circRNA can Encode MHC-I and MHC-II-Restricted Antigens to Induce CD8+ and CD4+ T Cell Responses, Respectively.

To test the ability of circRNA to elicit CD8+ and CD4⁺ T cell responses, we synthesized circRNA that encode model MHC-I-restricted SIINFEKL and MHC-II-restricted OVA323-339 (ISQ) epitopes. LNP-delivered circRNA-ISQ induced ISQ-specific CD4+ T cell responses in mice. Further, combined MHC-I circRNA-SIINFEKL and MHC-II circRNA-ISQ vaccines engaged both CD4⁺ effector/helper T cells and CD8⁺ effector T cells, thereby broadening and potentiating T cell responses (FIGS. 9A-B).

circRNA-Loaded Liposome and SM102-LNPs Showed High Tolerability.

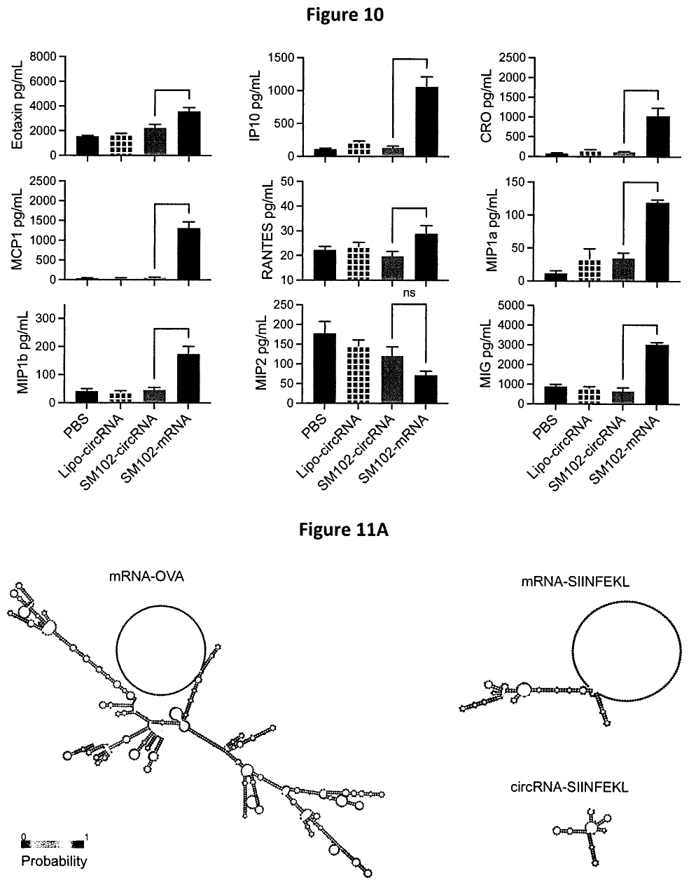

The reactogenicity and the associated tolerability of mRNA nanovaccines are among the major dose-lmiting factors. To study this for circRNA nanovaccines, we measured a panel of chemokines associated with the reactogenicity of RNA nanovaccines. Specifically, C57BL/6 mice were administered with circRNA-SIINFEKL-loaded liposome or SM102-LNPs, or traditional mRNA-OVA-loaded SM102-LNPs. Blood was collected 12 hours post administration. Luminex results of the serum cytokine/chemokine concentrations showed that liposomal and SM102-LNP circRNA showed significantly less reactogenicity than mRNA-OVA SM102-LNPs (FIG. 10), suggesting the high tolerability of liposomal and SM102-LNP circRNA vaccines.

Benchmarking circRNA to Current State-of-the-Art mRNA Vaccines.

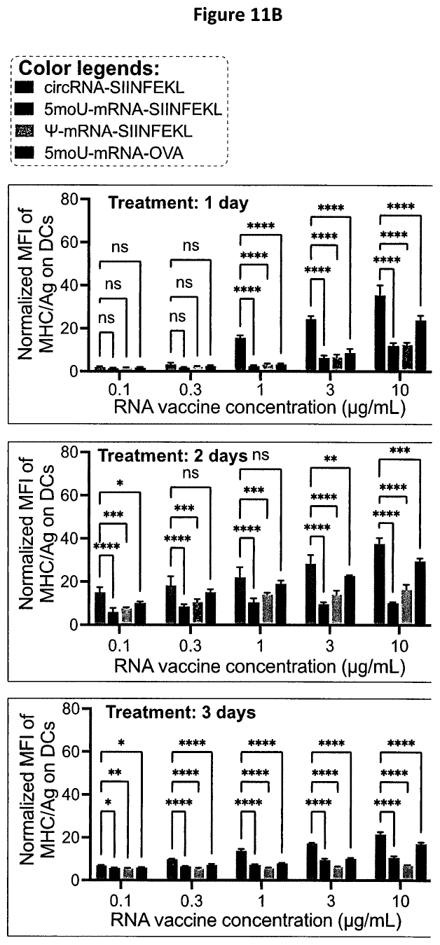

In DC2.4 cells treated with vaccines for 1-3 days, we measured (by flow cytometry) and compared the antigen presenting abilities by 1) circRNA vs. current state-of-the-art mRNA vaccines modified with 5′/3′ UTRs, polyA, CleanCap®, as well as 5 moU and pseudouridine (ψ), respectively (Trilink), and 2) circRNA encoding SIINFEKL vs. modified mRNA encoding SIINFEKL or OVA. Worth noting, in addition to the differences in modifications, these RNA also had drastic difference in their sizes, secondary structure complexity, as well as the length of double-stranded RNA that may cause adverse side effect such as activation of protein kinase K, illustrated in FIG. 11A. As a result, our circRNA was the most efficient in antigen presentation, evidenced by the highest levels of the MHC-I/SIINFEKL complex displayed by DCs (FIG. 11B). Collectively, these new observations further support our overall hypothesis and the concept of using nano-circRNA technology to develop novel melanoma immunotherapy.

### Example 2

Low-Dose Mini circRNA Encoding Tumor Antigens and Oncoviral Antigens for Antitumor Tumor Immunotherapy

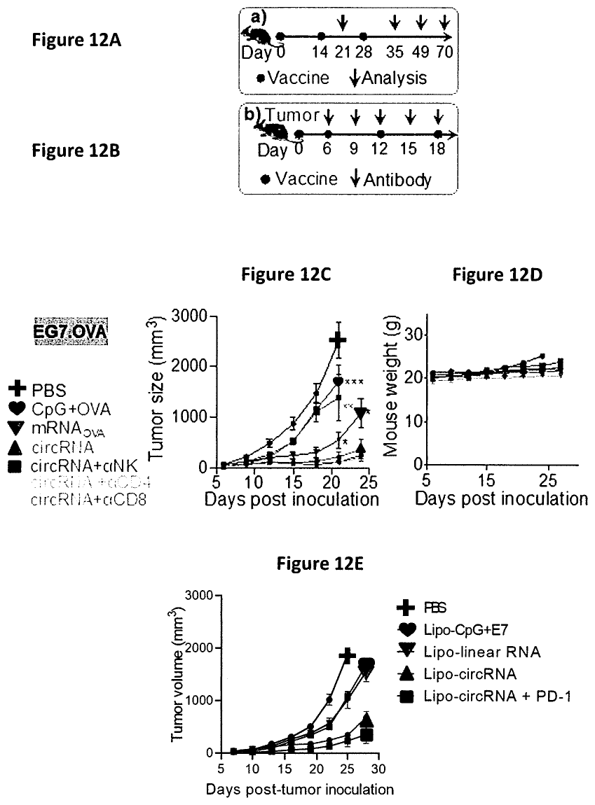

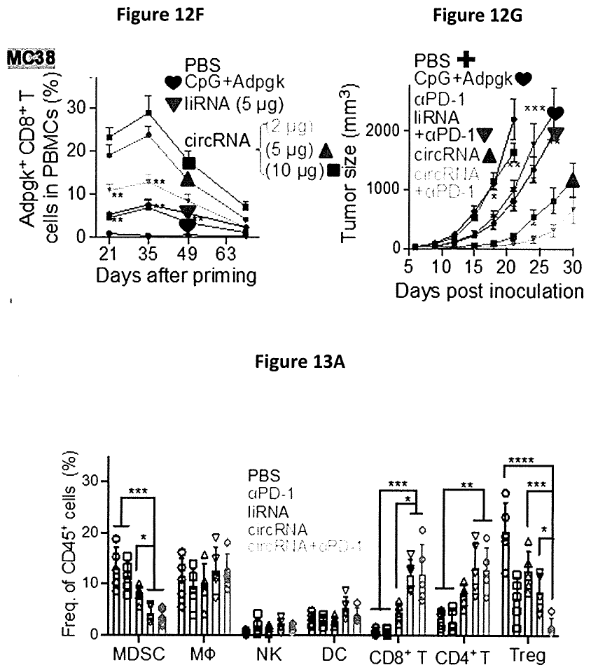

circRNA can be easily designed and synthesized to express customized antigen epitopes, including tumor neoantigens and oncoviral antigens. As above, we measured the T cell responses induced by these circRNA (FIG. 12A), and we also evaluated their tumor immunotherapeutic efficacy in target tumor-bearing syngeneic mice (FIG. 12B). For primary tumor models, female C57BL/6 mice (6-8 weeks; The Jackson Laboratory; n=6-8) were inoculated subcutaneously with 3×10⁵ EG7.OVA cells, MC38 cells, or TC-1 cells on the shoulder. When the tumor was established on day 6 (average tumor volume 40-60 mm³) post tumor inoculation, mice started to be treated with specified regimens by subcutaneous injection of Lipo-(CpG+peptide) (5 μg CpG and 10 μg OVA, Adpgk, or E7₄₃₋₆₂ peptide), Lipo-mRNA (5 μg OVA-mRNA), and Lipo-circRNA (5 μg circRNA). Mice were treated for 3 times every 3 days. Tumor tissues were collected to analyze the immune milieu in the tumor microenvironment. Tumor volume and mouse weight were monitored every 3 days. Mice were euthanized when any dimension of tumor was close to 2 cm or when mouse body weight was lost by over 20%. Tumor volume was calculated using the following formula:

Volume=(length*width²)/2

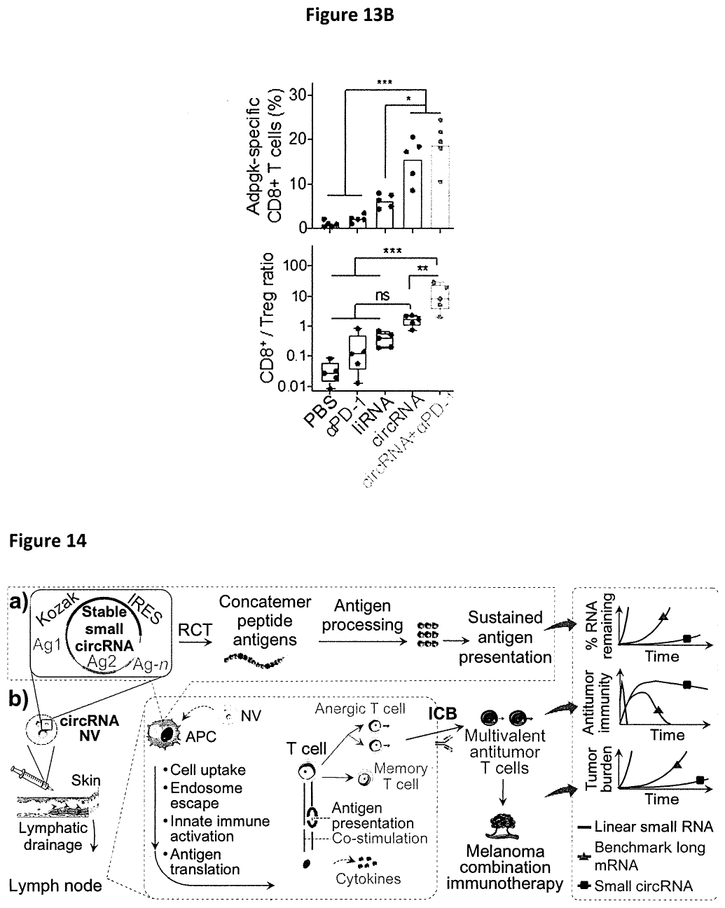

In lymphocyte depletion, female C57BL/6 mice (6-8 weeks) were inoculated subcutaneously with EG7.OVA or MC38 cells (3×10⁵) on the right shoulder. On day 6 when tumors were established, mice were divided into five groups having comparable tumor volumes (n=5). Five groups of mice were respectively vaccinated with PBS in group (1), and Lipo-circRNA (5 μg circRNA) in groups (2-5), by subcutaneous injection, in 50 μl PBS at the base of the tail on day 6, 12 and 18 post-tumor inoculation. Meanwhile, on days 6, 9, 12, 15, and 18 post-tumor inoculation, mice in groups (2-5) were also intraperitoneally injected with PBS in group (2), anti-CD4 in group (3), anti-CD8 in group (4), and anti-NK1.1 in group (5) (antibody dose: 200 μg per mouse). Tumor sizes and mouse weights were monitored every 3 days. Mice were killed when any tumor dimension was close to or already exceeded 2 cm. The tumor volume was calculated and analyzed as described above. The results are presented in FIG. 12C. The results showed that, in EG7.OVA tumor models, circRNA-SIINFEKL vaccines, alone or combined with anti-PD-1, significantly inhibited the progression of established tumors in a therapeutic setting, which outperformed CpG-adjuvanted peptide/protein vaccines, linear RNA vaccines, as well as modified protein (OVA)-encoding mRNA. Further depletion of CD8+ T cells, but not CD4+ T cells and NK cells, almost completely abrogated the therapeutic efficacy of circRNA vaccines, confirming the central role of CD8⁺ T cells in MHC-I-restricted circRNA-SIINFEKL. Of note, low-dose circRNA (3×5 μg) nanovaccines inhibited EG7.OVA tumor growth (see FIG. 13C for comparison) more effectively than nanovaccines of benchmark 5-methoxyuridine-modified CleanCap® mRNAOVA (TriLink) and CpG+OVA. Antibody depletion of CD8⁺ T cells, but not CD4⁺ T cells or natural killer (NK) cells, abrogated the therapeutic efficacy (FIG. 12C). Lastly, circRNA vaccines did not cause significant loss of mouse body weight, suggesting good safety (FIG. 12D). These data strongly support the use of circRNA nanovaccines for ICB combination cancer immunotherapy.

One type of antigens we tested using circRNA is oncoviral antigens, such as E7 derived from human papillomavirus (HPV). The ability of vaccines to elicit immune responses against such oncoviral antigens hold the potential to not only prophylactically protect the recipients from being infected with the corresponding viruses, but also treat preexisting infections and also treat cancers that are pathologically induced by the corresponding viral infections. We designed and synthesized circRNA for E7₄₃₋₆₂. In C57Bl/6 mice inoculated with E7-positive TC-1 tumor cells, circRNA-E7 effectively inhibited tumor growth, especially when combined with anti-PD-1 immune checkpoint inhibitor antibody (FIG. 12E).

Another type of antigens are tumor-specific neoantigens. We synthesized circRNA to encode a neoantigen called ADP-dependent glucokinase (Adpgk), which is MC38 tumor-derived MHC-I-restricted neoantigen. circRNA-Adpgk induced potent and dose-dependent Adpgk⁺ CD8⁺ T cell response (FIG. 12F). In C57Bl/6 mice inoculated with Adpgk-positive MC38 tumor cells, circRNA-Adpgk effectively inhibited tumor growth, especially when combined with anti-PD-1 immune checkpoint inhibitor antibody (FIG. 12G).

### Flow Cytometric Analysis of the MC38 Tumor Immune Microenvironment

For primary tumor models, female C57BL/6 mice (6-8 weeks. The Jackson Laboratory. n=6-8) were inoculated subcutaneously with 3×10⁵ MC38 cells on the shoulder. When the tumor was established on day 6 (tumor volume ˜40-60 mm³) post tumor inoculation, mice started to be treated with specified regimens by subcutaneously injection of Lipo-(CpG+Adpgk) (5 μg CpG and 10 μg Adpgk peptide), Lipo-LinearRNA (5 μg LinearRNA), Lipo-circRNA (5 μg circRNA), and Lipo-circRNA (5 μg circRNA)+200 μg anti-PD-1. Mice were treated for 2 times every 3 days. On day 12 following implantation, tumors were dissected from the surrounding fascia, weighed, mechanically minced, and treated with collagenase P (2 mg/ml, Sigma) and DNase I (50 μg/ml, Sigma) for 10 min at 37° C. Cells were passed through a 70-micrometre filter to remove clumps, diluted in medium, and a small aliquot taken directly for flow cytometry. Cell surface staining was performed with the indicated antibodies before fixation and permeabilization of the cells (Intracellular Fixation & Permeabilization Buffer Set, eBiosciences) for intracellular staining. All analysis was done with FlowJo software v 10.4.2 (FlowJo). The results are presented in FIGS. 13A-B. The results showed that relative to controls, circRNA alone or combined with anti-PD-1 significantly increase the tumor infiltration of CD8+ T cells (including antigen Adpgk-specific CD8+ T cells) and CD4+ T cells, while reducing the densities of immunosuppressive cells such as Treg and MDSC; further, the ratio of CD8+/CD4+ T cells, which predicts the tumor immunotherapeutic responses, were enhanced by circRNA alone or combined with anti-PD-1.

### Example 3

Nanoparticle Delivery of Bivalent Small Mini circRNA Vaccines for Melanoma Immunotherapy.

Melanoma is the most serious type of skin cancer. Although immune checkpoint blockade (ICB) immunotherapy has benefited many melanoma patients, there remains an unmet need as most patients do not respond to ICB. Cancer therapeutic vaccines can promote ICB therapeutic efficacy by generating or amplifying tumor-reactive T cells. Conventional cancer vaccines are associated with limitations such as low stability and bioavailability, preexisting anti-viral-vector immunity, weak antigenicity, or concerns over genomic integration or virulent reversion. mRNA vaccines hold a great potential for cancer immunotherapy, in part due to efficient delivery by nanocarriers. Yet, current mRNA vaccines rely on long linear mRNA that is associated with 1) limited biostability, despite extensive modifications, and the resulting limited shelf-life and moderate antigen translation efficiency, 2) complicated error-prone enzymatic production, and 3) limited loading capacity in nanocarriers.

To address these limitations, we have developed highly stable multivalent antigen-encoding mini circRNA as a novel type of mRNA vaccine (FIG. 14A) that can be efficiently loaded in nanoparticles directed to the host immune system to improve ICB-based melanoma immunotherapy (FIG. 14B). Small mini circRNA is comprised of minimal RNA elements to translate peptide antigens. Results presented in this example show that 1) chemically-defined mini circRNA was successfully synthesized by automated RNA synthesis prior to circularization; 2) mini circRNA has high loading capacity in nanocarriers and efficiently accumulated in lymph nodes and antigen-presenting cells in mice; 3) terminus-free circRNA, either free or loaded in nanoparticles, resists exonuclease degradation with enhanced stability in storage solutions (−20° C., 4° C., 23° C.) and in live cells, relative to current state-of-the-art modified mRNA vaccine; 4) circRNA vaccine is self-adjuvanted due to intrinsic RNA activation of intracellular pattern recognition receptors; 5) circRNA nanovaccines prolonged antigen translation with synchronized innate immunostimulation, which promotes T cell responses; 6) circRNA produces concatemer peptide antigens that, relative to minimal antigens, undergo proteolytic processing for the optimal antitumor T cell responses; and 7) remarkably, compared to the current state-of-the-art mRNA vaccines, low-dose small circRNA nanovaccines generate superior T cell immunity for improved antitumor efficacy in both young adult mice and immunosenescent aged mice. Our studies further showed that a bivalent melanoma nanovaccine elicited bi-specific T cell responses and significantly inhibited melanoma growth.

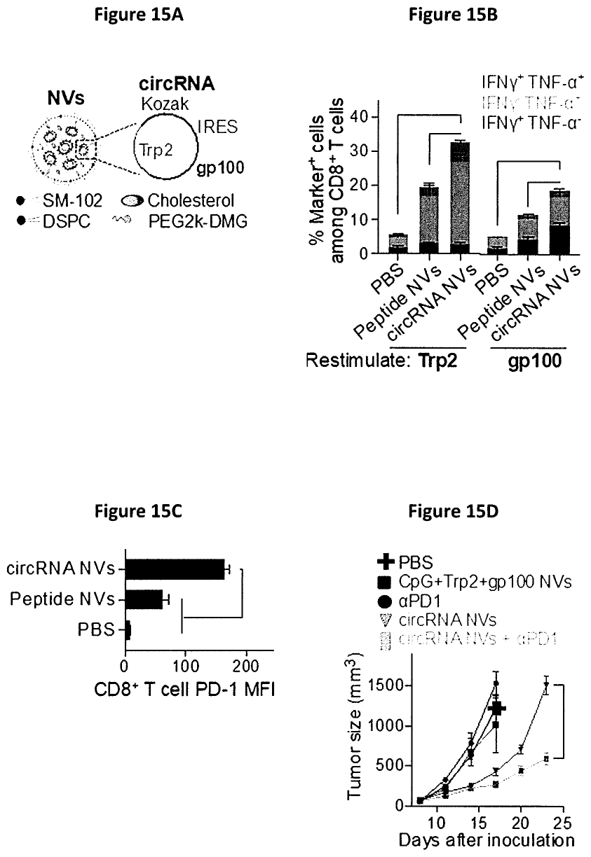

Melanoma antigen heterogeneity presents major hurdles for therapeutic vaccination. We and others showed heterogeneous melanoma antigen profiles, and bivalent melanoma-associated antigen Trp2/gp100 vaccine promoted melanoma therapeutic efficacy than monovalent ones. We synthesized codon-optimized circRNA for MHC-I-restricted murine Trp2₁₈₀₋₁₉₀ and human gp100₂₃₋₃₃ (FIG. 15A). Human gp100₂₃₋₃₃ is very immunogenic and primes T cells to recognize both human and murine gp100. Using SM-102 LNPs as carriers, circRNA-Trp2/gp100 nanovaccines induced bi-specific CD8⁺ T cell responses as shown by intracellular cytokine staining of CD8⁺ T cells from immunized mice (FIG. 15B), allowing it to overcome tumor heterogeneity and immune escape and hereby improving melanoma therapeutic efficacy. Intriguingly, circRNA nanovaccines upregulated immune checkpoint PD-1 on CD8⁺ T cells (FIG. 15C), providing a rationale for combining ICB with circRNA nanovaccines to achieve optimal antitumor efficacy. Indeed, in mice with poorly immunogenic B16F10 melanoma, while circRNA-Trp2/gp100 nanovaccines significantly retarded tumor growth, circRNA nanovaccines and αPD-1 combination further potentiated the therapeutic outcomes (FIG. 15D).

### Example 4

Synthesize and Characterize Mini circRNA for Tumor Antigens and Viral Antigens

Mini circRNA can be easily designed and synthesized to express customized antigen epitopes, as exemplified by the following mini circRNA vaccine against COVID-19.

### COVID-19 CircRNA Vaccine.

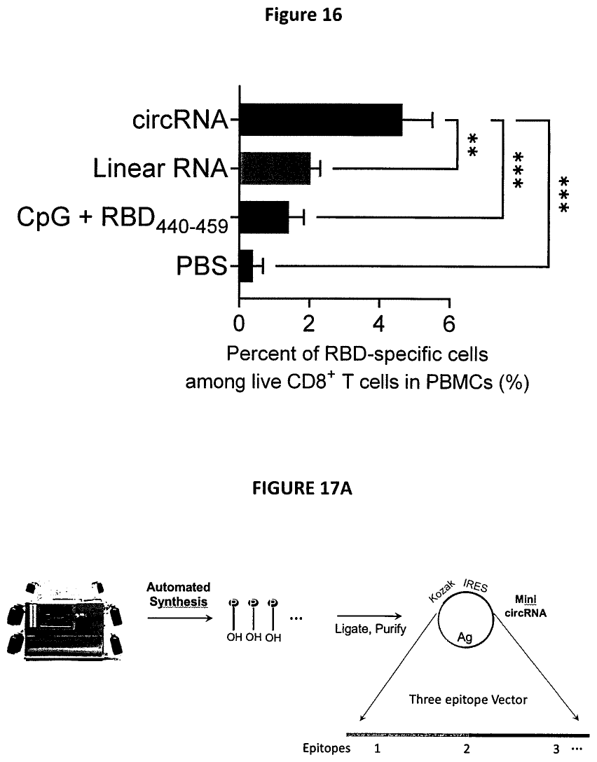

S-protein is a viral surface protein that mediates viral entry into host cells and is the most prominent target for vaccine development. Cellular immunity, including T cell responses, is also critical for the immune responses against SARS-CoV-2. Indeed, CD4⁺ and CD8⁺ T cells have been detected in 100% and 70%, respectively, of convalescent COVID patients. CircRNA was designed and synthesized to encode a Spike epitope, receptor-binding domain (RBD)₄₄₀₋₄₅₉. As above, we measured the T cell responses induced by these circRNA (see FIG. 12A). C57Bl/6 mice were immunized as above, and peripheral T cell responses were measured by tetramer staining as above (FIG. 16).

### Example 5

Rapid Manufacturing of Mini circRNA Vaccine Vectors

This example demonstrates the use of the platform technology provided by the invention, which allows rapid design and manufacture of the vaccine vectors. The immunogen(s) of interest may be rapidly swapped out for alternative immunogen(s) due to the modular structure and construction of the nucleotide identity. A first oligo incorporates a non-coding IRES and a Kozak sequence. Additional oligos are designed to encode the immunogen(s) of interest. The number of oligos depends on size and number of immunogens, but typically comprise 18 to 75 nts per peptide antigen. The vaccine vectors are formed by chemically ligating multiple synthetic single-stranded RNA oligonucleotides to form a circRNA. Not only does the method of the invention allow rapid production of vaccines, but it also produces a more effective vaccine, particularly vaccines against cancer cells.

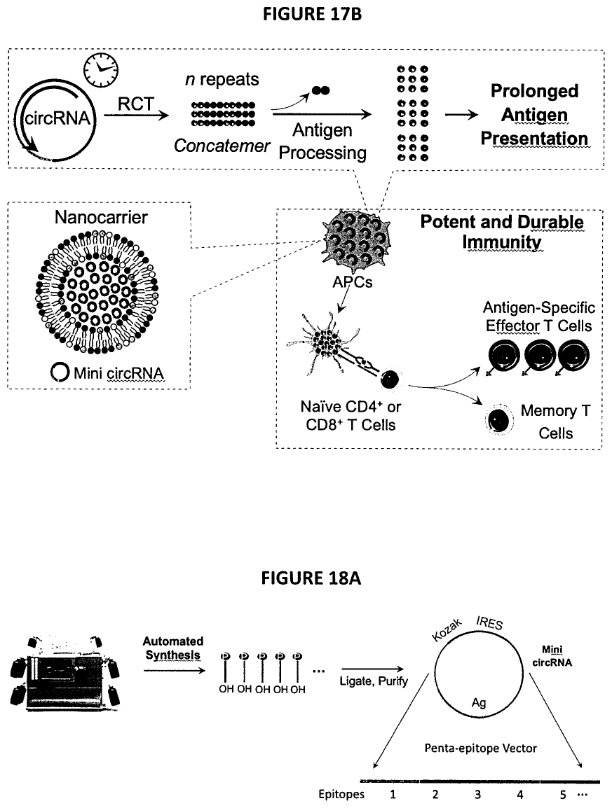

Synthetic single-stranded RNA oligonucleotides (oligos), typically having 40 to 150 nts, can be transcribed from a DNA template, but in this example the oligos are manufactured with automated synthesizer. FIG. 17A shows a flowchart of the process of oligo synthesis to produce a mixture of the desired oligos, which are then ligated chemically to form a mini circRNA comprising three epitopes. FIG. 17B illustrates the effects of the 3-epitope mini circRNA packaged in a nanocarrier and administered to a subject. The nanocarriers are taken up by antigen presenting cells (APCs) wherein the immunogens (antigens) are expressed by rolling concatemer transcription and are processed for presentation to naïve CD4+ or CD8+ T cells. Cells of the immune system, including antigen-specific effector T cells and memory T cells mount the desired immune response, thereby providing an effective vaccine.

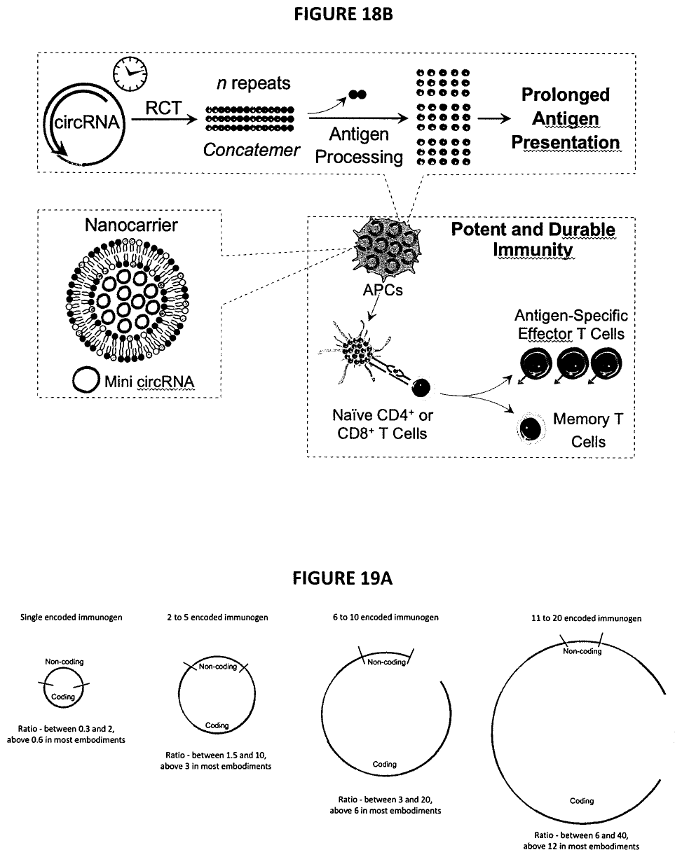

Since the oligos may be mass produced and stored, the first oligo may be reused for ligation with alternative immunogen-encoding oligos. FIG. 18A shows the construction process to form a pent-epitope vector comprising five immunogens. The first oligo having the Kozak and IRES sequences is identical to the one used to form the mini circRNA comprising three epitopes. FIG. 18B shows the effective immune response of a penta-epitope mini circRNA encapsulated in a nanocarrier and administered to a subject.

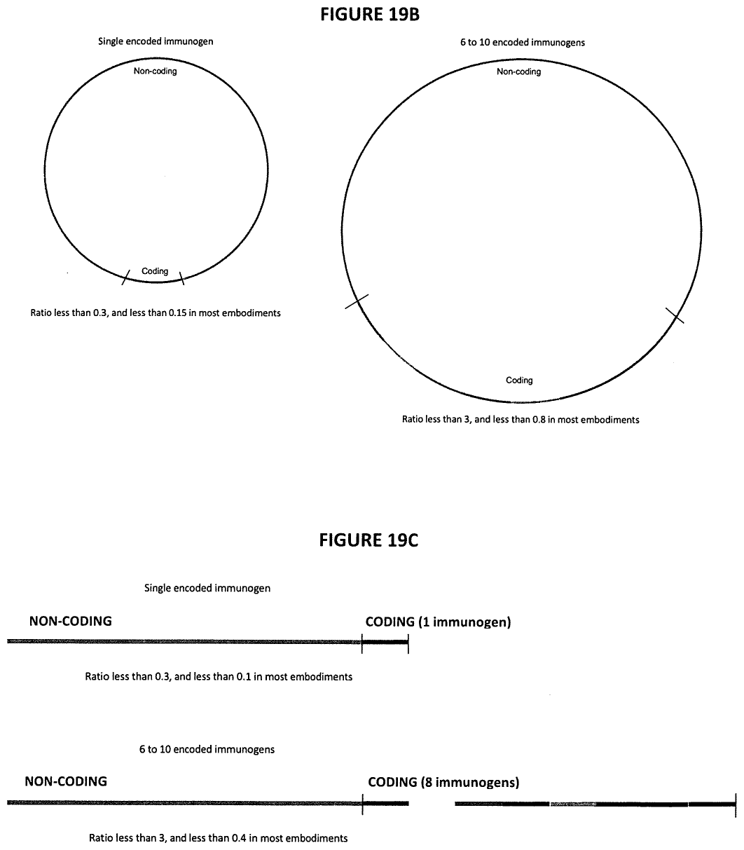

The ratio of coding to non-coding regions is a key feature that allows delivery and expression of antigen(s) at dramatically higher levels than can be achieved with traditional RNA vaccine vectors. The minimal non-coding region unexpectedly is highly effective and efficient at engaging ribosomes to initiate and maintain cap-independent translation. FIG. 19A illustrates additional examples of mini circRNA that may be synthesized using the steps of the process shown in FIGS. 17A and 18A. In a circRNA having a single encoded immunogen, the ratio of coding to non-coding is usually between 0.33 and 2 and is higher than 0.67 in most embodiments. A circRNA having 2 to 5 encoded immunogens will have a ratio between 1.67 to 10 and is higher than 3.33 in most embodiments. A circRNA having 6 to 10 encoded immunogens will have a ratio between 3.33 and 20 and is higher than 6.67 in most embodiments. A circRNA having 11 to 20 encoded immunogens will have a ratio between 6.67 and 40 and is higher than 13.33 in most embodiments. In contrast, FIG. 19B illustrates the ratio of coding to non-coding is significantly lower when using the same coding regions. In the illustrated example, the ratio is always lower than 3, and is frequently less than 1. FIG. 19C illustrates the ratio for a linear mRNA construct having the same immunogens, similar to the circRNA ratios shown in FIG. 19B.

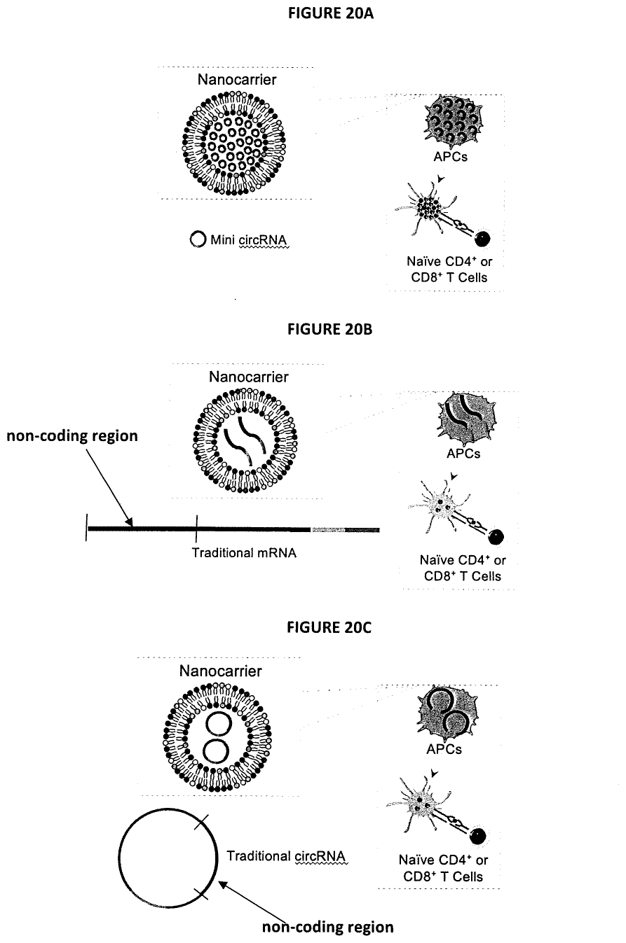

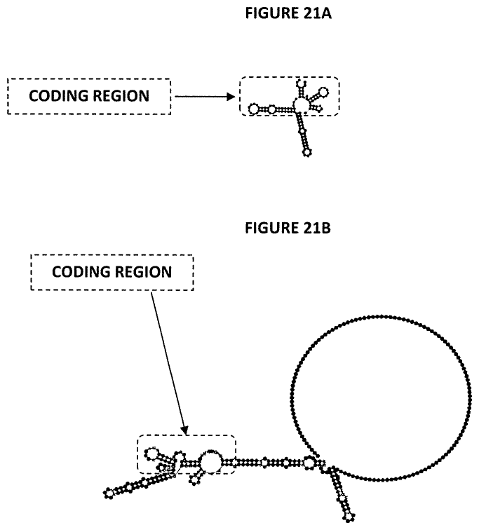

FIG. 20A illustrates the contrast between the effects of a mini circRNA vaccine and two forms of “traditional” RNA vaccines, shown in FIGS. 20B and 20C. Traditionally, a long untranslated region with IRES containing significant secondary structure was thought to be not only optimal but also required for efficient translation and immunogenicity. Traditional mRNA vectors have the largest untranslated sequences, including a 5′ cap and untranslated region, a 3′ untranslated region, and a polyA tail. Traditional mRNA is also not as biostable as circular RNA and does not survive as long in APSs, resulting in much lower immunogenicity. However, the size requirements of traditional RNA vaccines means that at least 10-fold fewer copies of coding RNA can be delivered to APCs, thus, a less robust immunogenic response can be achieved than that of the mini circRNA. The mini circRNA of the invention restricts the size of the non-coding region for cap-independent translation. This advantageously maximizes the coding RNA density within a nanocarrier and permits delivery to APCs while minimizing the amount of exposure to total RNA and lipid. Since RNA and lipid are both activators of innate immune sensors (such as TLRs), a traditional linear mRNA and a traditional circRNA risk over-activation of the immune system, which in turn reduces transfection and expression. FIGS. 19B and 19C illustrate the ratios of coding to non-coding regions in minicircular RNA, traditional mRNA and traditional circular RNA vectors. Mini circRNA vectors of the invention have non-coding regions of less than 300 nts, and in most embodiment, the range is 50 to 100 nts. In contrast, tradition mRNA vectors have non-coding regions of greater than 1,000 nts, and traditional circRNA vectors traditionally have non-coding regions of greater than 300 nts, and more commonly these are in the range of 500 to 1,500 nts. The mini cicrRNA remains highly self-adjuvanting and substantially increases the antigen expression and presentation in APCs. This is particularly an advantage for a vaccine against a cancer, since the immune response must be very high and sustained, compared to a vaccine against an infectious agent.

Finally, FIGS. 21A and 21B are predictive illustrations of 3-D structure and size differences between a mini circRNA of the invention and a traditional mRNA, each comprising nucleotides encoding the same epitope. The mini circRNA comprises only a minimal non-coding region, which forms a small hairpin structure illustrated predominately in red. The smaller size allows more copies of the mini circRNA to be packaged in carriers and delivered to cells in a subject who is a recipient of the vaccine. The traditional mRNA comprises regulatory elements in non-coding regions that form extensive hairpin and loop structures, which are illustrated predominantly in red. These additional 3-D structures limit the copy number that can be packaged into carriers and delivered to cells and increase the chances of over-stimulation of the innate immune system and rapid degradation of the mRNA before an appropriately specific immune response can be mounted. These structures also make the traditional mRNA molecule more vulnerable to surveillance and nuclease proteins in the host cells, thus contributing to the limited half-life of traditional mRNA vaccines.

### Example 6

Rapid Manufacturing of Mini circRNA Vaccine Vectors

This example demonstrates the use of the platform technology provided by the invention, which allows rapid design and manufacture of the vaccine vectors. The immunogen(s) of interest may be rapidly swapped out for alternative immunogen(s) due to the modular structure and construction of the nucleotide identity. A first oligo incorporates a non-coding IRES and a Kozak sequence. Additional oligos are designed to encode the immunogen(s) of interest. The number of oligos depends on size and number of immunogens, but typically comprise 18 to 75 nts per peptide antigen. The vaccine vectors are formed by chemically ligating multiple synthetic single-stranded RNA oligonucleotides to form a circRNA. Not only does the method of the invention allow rapid production of vaccines, but it also produces a more effective vaccine, particularly vaccines against cancer cells.

Synthetic single-stranded RNA oligonucleotides (oligos), typically having 40 to 150 nts, can be transcribed from a DNA template, but in this example the oligos are manufactured with automated synthesizer. FIG. 18A shows a flowchart of the process of oligo synthesis to produce a mixture of the desired oligos, which are then ligated chemically to form a mini circRNA comprising three epitopes. FIG. 18B illustrates the effects of the 3-epitope mini circRNA packaged in a nanocarrier and administered to a subject. The nanocarriers are taken up by antigen presenting cells (APCs) wherein the immunogens (antigens) are expressed by rolling concatemer transcription and are processed for presentation to naïve CD4+ or CD8+ T cells. Cells of the immune system, including antigen-specific effector T cells and memory T cells mount the desired immune response, thereby providing an effective vaccine.

Since the oligos may be mass produced and stored, the first oligo may be reused for ligation with alternative immunogen-encoding oligos. FIG. 19A shows the construction process to form a pentaepitope vector comprising five immunogens. The first oligo having the Kozak and IRES sequences is identical to the one used to form the mini circRNA comprising three epitopes. FIG. 19B shows the effective immune response of a penta-epitope mini circRNA encapsulated in a nanocarrier and administered to a subject.

The ratio of coding to non-coding regions is a key feature that allows delivery and expression of antigen(s) at dramatically higher levels than can be achieved with traditional RNA vaccine vectors. The minimal non-coding region unexpectedly is highly effective and efficient at engaging ribosomes to initiate and maintain cap-independent translation. FIG. 20A illustrates additional examples of mini circRNA that may be synthesized using the steps of the process shown in FIGS. 17A and 18A. In a circRNA having a single encoded immunogen, the ratio of coding to non-coding is usually between 0.33 and 2 and is higher than 0.67 in most embodiments. A mini circRNA having 2 to 5 encoded immunogens will have a ratio between 1.67 to 10 and is higher than 3.33 in most embodiments. A mini circRNA having 6 to 10 encoded immunogens will have a ratio between 3.33 and 20 and is higher than 6.67 in most embodiments. A mini circRNA having 11 to 20 encoded immunogens will have a ratio between 6.67 and 40 and is higher than 13.33 in most embodiments. The relative size of immunogen encoding sequence and non-coding region sequence in FIG. 19B for a traditional circular RNA and in FIG. 19C showing a traditional linear mRNA encoding a single immunogen, with the ratio of non-coding to coding regions less than 0.3, and less than 0.1 in most instances. The ratio of non-coding to coding regions when comprising 6-10 immunogens is less than 3, and less than 0.4 in most instances. In any instance, the ratio of a mini circRNA is lower than traditional forms carrying the same payload of immunogenic sequences in the coding regions.

FIGS. 20A-C show comparisons of the number of copies that can be formulated within nanoparticle carriers. Due to the small size of the mini circRNA, the number of copies of any given immunogen is greater than can be achieved with traditional linear mRNA or circRNA, thus providing, in some cases, at least an order of magnitude greater number of copies. This feature allows the mini circRNA vaccine vector of the invention to induce a robust and sustained immune response that is required for many methods of treatment, which is particularly applicable to treatment of cancers.

More importantly, FIGS. 20A-C also illustrate the contrast between the effects of administering a mini circRNA vaccine and two forms of “traditional” RNA vaccines. FIG. 20B shows a traditional circular RNA, such as can be used as a delivery vector for a vaccine or therapeutic. It should also be noted that a naturally occurring circular RNA typically comprises an extensive amount of non-coding sequences, such as that shown in the single encoded immunogen in FIG. 20B. FIG. 20C shows the typical composition of traditional mRNA molecules and/or vaccine vectors. Traditionally, a long untranslated region with IRES containing significant secondary structure was thought to be not only optimal but also required for efficient translation and immunogenicity. Traditional mRNA vectors have the largest untranslated sequences, including a 5′ cap and untranslated region, a 3′ untranslated region, and a poly A tail. Traditional mRNA is also not as biostable as circular RNA and does not survive as long in APSs, resulting in much lower immunogenicity. However, the size requirements of traditional RNA vaccines means that at least 10-fold fewer copies of coding RNA can be delivered to APCs, thus, a less robust immunogenic response can be achieved than that of the mini circRNA shown in FIG. 20A. The mini circRNA of the invention restricts the size of the non-coding region for cap-independent translation. This advantageously maximizes the coding RNA density within a nanocarrier and permits delivery to APCs while minimizing the amount of exposure to total RNA and lipid. Since RNA and lipid are both activators of innate immune sensors (such as TLRs), a traditional linear mRNA and a traditional circRNA risk over-activation of the immune system, which in turn reduces transfection and expression.

FIGS. 21A-C provide a further illustration of the ratios of coding to non-coding regions in minicircular RNA, traditional mRNA and traditional circular RNA vectors. Mini circRNA vectors of the invention typically can have non-coding regions of less than 300 nts, and in most embodiment, the range is 50 to 100 nts. In contrast, tradition mRNA vectors have non-coding regions of greater than 1,000 nts, and traditional circRNA vectors traditionally have non-coding regions of greater than 300 nts, and more commonly these are in the range of 500 to 1,500 nts. The mini circRNA remains highly self-adjuvanting and substantially increases the antigen expression and presentation in APCs. This is particularly an advantage for a vaccine against a cancer, since the immune response must be very high and sustained, compared to a vaccine against an infectious agent.

Finally, FIGS. 22A and 22B are predictive illustrations of 3-D structure and size differences between a mini circRNA of the invention and a traditional mRNA, each comprising nucleotides encoding the same epitope. The mini circRNA comprises only a minimal non-coding region, which forms a small hairpin structure illustrated predominately in red. The smaller size allows more copies of the mini circRNA to be packaged in carriers and delivered to cells in a subject who is a recipient of the vaccine. The traditional mRNA comprises regulatory elements in non-coding regions that form extensive hairpin and loop structures, which are illustrated predominantly in red. These additional 3-D structures limit the copy number that can be packaged into carriers and delivered to cells and increase the chances of over-stimulation of the innate immune system and rapid degradation of the mRNA before an appropriately specific immune response can be mounted. These structures also make the traditional mRNA molecule more vulnerable to surveillance and nuclease proteins in the host cells, thus contributing to the limited half-life of traditional mRNA vaccines.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

We claim:

1. A mini circular RNA (circRNA) vaccine vector comprising 30 to 3300 nucleotides, constructed with 1 to 40 synthetic single-stranded oligonucleotide RNA sequences ligated together to form the mini circRNA vaccine vector, comprising
a non-coding region comprising an internal ribosome entry site (IRES) and/or a Kozak sequence,
a coding region comprising nucleotide sequences encoding at least one immunogen, and optionally comprising nucleotide sequences encoding one or more linkers or spacers.

2. The mini circRNA vaccine vector of claim 1, wherein at least one immunogen induces cell-mediated immunity in at least one cell type selected from the group consisting of immunogen-specific CD4⁺ T cells and immunogen-specific CD8⁺ killer T cells.

3. The mini circRNA vaccine vector of claim 1, wherein ligation does not introduce extraneous RNA fragments containing dsRNA

4. The mini circRNA vaccine vector of claim 1, wherein there is no STOP codon immediately following the open reading frame of the coding region

5. The mini circRNA vaccine vector of claim 1, wherein each of the 1 to 40 synthetic single-stranded oligonucleotide RNA sequences are in the range of 40 to 150 nucleotides in length.

6. The mini circRNA vaccine vector of claim 1, wherein the non-coding region comprises 300 or fewer nucleotides.

7. The mini circRNA vaccine vector of claim 1, wherein the non-coding region comprises 150 or fewer nucleotides.

8. The mini circRNA vaccine vector of claim 1, wherein the nucleotide sequences that encode the one or more linkers or spacers further encodes a peptide cleavage site or structural peptide linker.

9. The mini circRNA vaccine vector of claim 1, wherein the IRES is selected from the group consisting of LINE1, crTMV, Rbm3, and human c-MYC.

10. The mini circRNA vaccine vector of claim 1, wherein the at least one immunogen is selected from the group consisting of a tumor associated antigen, a tumor neoantigen, an oncoviral antigen and a testis cancer antigen.

11. The mini circRNA vaccine vector of claim 1, wherein the coding region encodes a plurality of peptide immunogens, wherein the peptide immunogens are separated by linkers comprising peptide cleavage sites or structural peptide linkers.

12. The mini circRNA vaccine vector of claim 1, wherein the coding region comprises a nucleotide sequence encoding a plurality of peptide immunogens positioned consecutively with no peptide cleavage site or structural linker, wherein multiple immunogens are translatable into a peptide concatemer.

13. The mini circRNA vaccine vector of claim 1, wherein the mini circRNA is encapsulated in a nano-carrier selected from the group consisting of a liposome, an exosome, a nanoparticle and a lipid nanoparticle.

14. A method of treating cancer in a subject in need thereof, comprising the steps of
identifying a cancer antigen expressed in cells of the cancer,
synthesizing a mini circular RNA (circRNA) vaccine vector wherein a coding region comprises nucleotide sequences encoding at least a portion of the cancer antigen and a non-coding region comprises at least one regulatory element, and
administering a therapeutically effective amount of the circRNA vaccine vector to the subject intramuscularly, intravenously, subcutaneously, intradermally, intranasally, or intratracheally,
wherein the therapeutically effective amount is sufficient to induce an immune response in the subject.

15. The method of claim 14, wherein the administering step is repeated at intervals of 1 to 8 weeks.

16. The method of claim 14, wherein the mini circRNA vaccine vector is administered in combination with an immunotherapy agent selected from the group consisting of a PD-1 inhibitor and PD-L1 inhibitor.

17. The method of claim 14, wherein the at least one immunogen is HLA-matched to the subject.

18. The method of claim 14, wherein the coding region comprises a nucleotide sequence encoding at least one immunogen selected from the group consisting of a mutant KRAS antigen, a melanoma tumor-specific antigen and an isocitrate dehydrogenase tumor-specific antigen.

19. A method of preparing a self-adjuvanted mini circular RNA (circRNA) for a subject in need thereof, comprising 80 to 2000 nucleotides with at least one translation initiation element, and encoding at least one immunogen, wherein the circRNA is synthesized by the steps of
providing one or more DNA scaffold or splints;
synthesizing one or more linear single-stranded RNA oligonucleotides;
hybridizing the one or more DNA scaffold or splint with at least one of the one or more linear single-stranded RNA oligonucleotides or with a plurality of the one or more linear single-stranded RNA nucleotides so as to bring 5′ and 3′ ends of the at least one of the one or more linear single stranded oligonucleotides or 5′ and 3′ ends of at least two of the plurality of one or more linear single-stranded RNA oligonucleotides within close proximity of each other;
ligating the 5′ and 3′ ends of the at least one single-stranded RNA oligonucleotide or the 5′ and 3′ ends of the two of the plurality of the one or more linear single-stranded RNA oligonucleotides to form the mini circRNA vaccine;
removing the at one or more DNA scaffold or splints;
purifying the mini circRNA; and
formulating the mini circRNA in a pharmaceutically acceptable carrier.
